(19) United States
(12) Patent Application Publication
(10) Pub. No.: US 2012/0172453 A1
(43) Pub. Date: Jul. 5, 2012

(54) METHODS FOR TREATING NEURODEGENERATIVE DISEASES

(75) Inventors: Ben A. Barres, Palo Alto, CA (US); Naoki Nakayama, Houston, TX (US); Hiroaki Serizawa, Palo Alto, CA (US); Ankush B. Argade, Foster City, CA (US)

(73) Assignee: Coyote Pharmaceuticals, Inc.

(21) Appl. No.: 13/223,244

(22) Filed: Aug. 31, 2011

Related U.S. Application Data
(60) Provisional application No. 61/379,316, filed on Sep. 1, 2010.
(60) Provisional application No. 61/510,002, filed on Jul. 20, 2011.

(51) Int. Cl. A61K 31/121; C07C 29/147; C07C 45/65; C07D 317/12; A61P 25/28; A61P 25/00; C12N 5/0793; C07C 49/203; C07C 45/59

(52) U.S. Cl. 514/675; 568/417; 568/884; 568/388; 568/386; 435/375; 549/430

(57) ABSTRACT

This invention relates to the 5-cis and 5-trans isomers of geranylgeranyl acetone, preferably such synthetic isomers, and pharmaceutical compositions containing such isomers. Other aspects of this invention relate to the use of geranylgeranyl acetone and its isomers in methods for inhibiting neural death, increasing neural activity, and increasing axon growth and cell viability. Geranylgeranyl acetone is a known anti-ulcer drug used commercially and in clinical situations. GGA has also been shown to exert cytoprotective effects on a variety of organs, such as the eye, brain, and heart.

### CROSS-REFERENCE TO RELATED APPLICATIONS

This application claims priority under 35 U.S.C. §119(e) to U.S. Provisional Application Ser. Nos. 61/379,316 filed Sep. 1, 2010, and 61/510,002 filed Jul. 20, 2011 each of which is incorporated herein by reference in its entirety.

### FIELD OF THE INVENTION

This invention relates generally to methods for inhibiting neural death and increasing neural activity with the compound geranylgeranyl acetone (GGA), and compositions used for these indications. The invention also relates to cis and trans isomers of geranylgeranyl acetone, and mixtures of various GGA isomers and their therapeutic uses.

### STATE OF THE ART

Geranylgeranyl acetone is an acyclic isoprenoid compound with a retinoid skeleton that has been shown to induce expression of heat shock proteins in various tissue types. GGA is a known anti-ulcer drug used commercially and in clinical situations.

GGA has also been shown to exert cytoprotective effects on a variety of organs, such as the eye, brain, and heart (See for example Ishii Y., et al., Invest Ophthalmol Vis Sci 2003; 44:1982-92; Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 104:1837-43; and Suzuki S, et al., Kidney Int 2005; 67:2210-20). The effects and cytoprotective benefits of GGA in these settings is less understood as is the relationship of isomers of GGA to these cytoprotective benefits. Of particular interest, is the effect of GGA on extranuclear neurodegeneration both on an intracellular or extracellular basis.

Neurodegeneration is often the result of increased age, sporadic mutations, disease, and/or protein aggregation in neural cells. Neurodegenerative diseases are often characterized by a progressive neurodegeneration of tissues of the nervous system and a loss of functionality of the neurons themselves. One commonality seen in most neurodegenerative diseases is the accumulation of protein aggregates intracellularly or in the extracellular space between neurons.

Protein aggregation is facilitated by partial unfolding or denaturation of cellular proteins. This may be due to mutations in the sequence of the DNA, transcriptional misincorporation, modifications to the RNA, and modifications or oxidative stress to the protein. There is an increasing amount of evidence to suggest that protein aggregates contribute to disease progression. In one study, aggregates of two non-disease proteins were formed in vitro and added to the medium of cultured cells. Addition of granular-structured, protein aggregates significantly reduced the cell viability of both the fibroblastic cell line (NIH-3T3) and neural cell line (PC12). However, addition of more organized fibrillar protein aggregates did not compromise the cell viability. (Bucciantini et al. (2002) Nature 14:507-510.)

Protein aggregates can be extracellular (i.e. in the space between neural cells), intracellular such as intranuclear (i.e. in the nucleus of the cell), or in the cytoplasm. Extracellular and/or cytoplasm protein aggregates are a pathological characteristic of Alzheimer's disease (AD) and amyotrophic lateral sclerosis (ALS). AD is a progressive brain disease that destroys memory and cognitive function. AD has been linked to the aggregation of the β-amyloid peptide. The β-amyloid peptide is derived from the amyloid precursor protein (APP) that has been processed by two aspartyl proteases called β and γ secretases. Similar to AD, ALS is also a progressive neurodegenerative disease and is characterized by loss of functionality of motor neurons. The progressive degeneration of motor neurons results in loss of ability of the brain to initiate and control muscle movement. ALS is a devastating disease, in which the last stage is complete paralysis. The complete molecular mechanism of disease progression in ALS is not yet clear, but mutations in the Cu/Zn superoxide dismutase (Sod) gene, Sod1, have been linked to the degeneration of motor neurons. The disease symptoms of ALS and AD may differ, but the presence of cytotoxic aggregate proteins in both diseases suggests a common mechanism in pathogenicity. (Ross & Poirier. (2004) Nat. Med. ppS10-S17; Irvine et al. (2008) Mol. Med. 14(7-8):451-464; Wang et al. (2008) PLoS One Vol. 6, Issue 7, pp 1508-1526. Iguchi et al. (2009) J. Bio Chem. Vol. 284 no. 33 pp. 22059-22066.)

Recently, it was also found that depletion of the TDP-43 protein (TAR DNA binding protein or TARDBP) in Neuro-2a cells causes protein aggregation similar to what is observed in ALS. In fact, point mutations in TARDBP have been linked to familial and sporadic ALS. TDP-43 depletion by TARDBP siRNA in Neuro-2a cells also causes inhibition of the biological activity of the Rho family of small G proteins. Therefore, TDP-43 and Rho family proteins negatively affect protein aggregate formation in neural cells. The Rho family proteins are responsible for regulating cell movement, cell survival, cell growth, transcription, and motility of cells (Iguchi et al. (2009) J. Bio Chem. Vol. 284 no. 33 pp. 22059-22066). Therapies that prevent reduction in the amount and/or activity of TDP-43 or Rho family proteins may have a neuroprotective effect on cells.

There is a need for more effective therapies for neurodegenerative diseases such as AD and ALS. Research suggests that therapies targeting cellular mechanisms that control protein aggregation are likely to reduce the loss of functionality and viability of neurons in these diseases, thus, alleviating the symptoms. Therapies that enhance a small G protein activity may also be useful in inhibiting neural death and increasing neural activity in ALS. This application relates to the use of geranylgeranyl acetone (GGA) to inhibit or alter the formation of protein aggregates and modulate the activity of small G proteins in neural cells.

### SUMMARY OF THE INVENTION

This invention relates to pharmaceutical uses of geranylgeranyl acetone, compounds and pharmaceutical compositions of isomers of geranylgeranyl acetone, preferably synthetic geranylgeranyl acetone and methods of using such compounds and pharmaceutical compositions. In addition, this invention relates to methods of preparing GGA, and its isomers. In certain aspects, this invention relates to a 5-trans isomer compound of formula I:

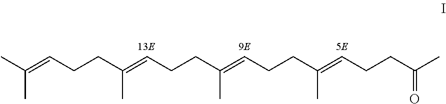

wherein I is at least 80% in the 5E, 9E, 13E configuration. In one embodiment, this invention provides a compound, which is synthetic 5E, 9E, 13E geranylgeranyl acetone. In another embodiment, the synthetic 5E, 9E, 13E geranylgeranyl acetone is free of 5Z, 9E, 13E geranylgeranyl acetone. In another aspect, this invention provides a pharmaceutical composition comprising synthetic GGA or synthetic 5E, 9E, 13E GGA, and at least one pharmaceutical excipient.

In another aspect, this invention provides a composition for increasing the expression and/or release of one or more neurotransmitters from a neuron at risk of developing pathogenic protein aggregates associated with AD or ALS, said composition comprising a protein aggregate inhibiting amount of GGA, or an isomer or a mixture of isomers thereof.

In another aspect, this invention provides a composition for increasing the expression and/or release of one or more neurotransmitters from a neuron at risk of developing extracellular pathogenic protein aggregates, said composition comprising an extracellular protein aggregate inhibiting amount of GGA, or an isomer or a mixture of isomers thereof.

Another aspect of this invention relates to a synthetic 5-cis isomer compound of formula II:

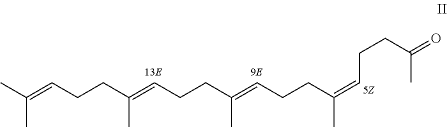

wherein II is at least 80% in the 5Z, 9E, 13E configuration, or a ketal thereof of formula XII:

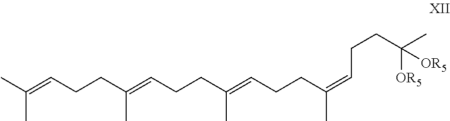

wherein each R₅ independently is C₁-C₆ alkyl, or two R₅ groups together with the oxygen atoms they are attached to form a 5 or 6 membered ring, which ring is optionally substituted with 1-3, preferably 1-2, C₁-C₆ alkyl groups. Preferably, the two R₅ groups are the same. In one embodiment, R₅ is, methyl, ethyl, or propyl. In another embodiment, the cyclic ring is:

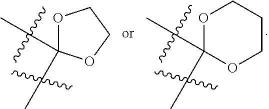

In one of its method aspects, there is provided a method comprising one or more of the following steps. Reacting compound of formula III under halogenation conditions to give a compound of formula IV. Reacting the compound of formula IV with alkyl acetoacetate of formula R¹OOCCH₂COCH₃, wherein R¹ is alkyl, under alkylation conditions to provide compound of formula V as a mixture of R and S enantiomers. Reacting compound of formula V under a Hydrolysis and decarboxylation of condition to give compound of formula VI.

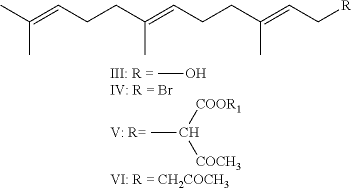

Reacting a compound of formula VI with compound of formula VII, wherein R₂ and R₃ independently are alkyl, under olefination conditions to stereoselectively provide a compound of formula VIII. Reacting a compound of formula VIII under reduction conditions to provide a compound of formula IX. Halogenations of compound of formula IX under conditions to give a compound of formula X. Reacting the compound of formula X with alkyl acetoacetate, under alkylation conditions to provide compound of formula XI as a mixture of R and S enantiomers. Reacting compound with formula XI under hydrolysis and decarboxylation of condition to give a compound of formula I.

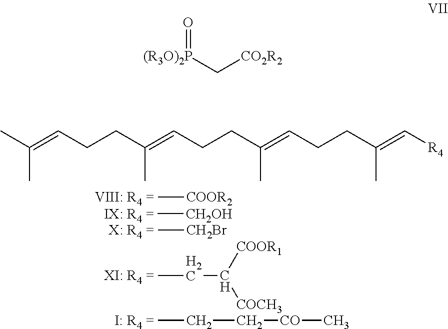

In another modification, this invention provides the method of decarboxylating the compound of formula XIA or a salt thereof, wherein R₄ is —CH₂—CH(COCH₃)CO₂H.

In another of its method aspects, there is provided a method for increasing the axon growth of neurons by contacting said neurons with an effective amount of GGA.

In another aspect, this invention relates to a method for inhibiting or reducing the cell death of neurons susceptible to neuronal cell death, which method comprises contacting said neurons with an effective amount of GGA.

In yet another of its method aspects, there is provided a method for increasing the neurite growth of neurons by contacting said neurons with an effective amount of GGA.

Other aspects of this invention relate to methods for neurostimulation by contacting neurons with an effecting amount of GGA. In one embodiment neurostimulation consists of increasing the expression and/or release of one or more neurotransmitters from a neuron. In another embodiment the neurostimulation consists of enhancing synapse formation of a neuron, or, alternatively, enhancing electrical excitability. In yet another embodiment, the neurostimulation includes modulating the activity of G proteins in neurons. In a related embodiment, the activation of G proteins is enhanced by GGA.

In another embodiment, this invention provides methods for neuroprotection of neurons at risk of neural damage or death by contacting said neurons with an effective amount of GGA. In one particular embodiment, neurons at risk of neural toxicity or death include those affected by, or those in the pathogenesis of, Alzheimer's Disease or ALS. In each case, neuroprotection is affected by contacting the neurons at risk of neural damage or death with an effective amount of GGA.

Yet another aspect of this invention relates to neuroprotective methods such as methods for protecting neurons at risk of neurotoxicity wherein the method comprises contacting cells comprising the neurons at risk of neurotoxicity with an effective amount of GGA. Without being limited to a particular theory, it is contemplated that GGA may be antagonistic to the neurotoxicity of the β-amyloid peptide or oligomers or polymers thereof.

Yet another neuroprotective aspect is a method for protecting neurons from neurodegeneration arising from ALS.

In another aspect, this invention relates to a method for inhibiting the death of neurons due to formation of or further formation of pathogenic protein aggregates either between, outside or inside neurons, wherein said method comprises contacting said neurons at risk of developing said pathogenic protein aggregates with a protein aggregate inhibiting amount of GGA provided that said pathogenic protein aggregates are not related to SBMA.

In yet another aspect, this invention relates to a method for inhibiting neural death and increasing neural activity in a mammal suffering from a neural disease, wherein the etiology of said neural disease comprises formation of protein aggregates which are pathogenic to neurons which method comprises administering to said mammal an amount of GGA which will inhibit further pathogenic protein aggregation provided that said pathogenic protein aggregation is not intranuclear.

Another aspect of this invention relates to a method for inhibiting neural death and increasing neural activity in a mammal suffering from a neural disease, wherein the etiology of said neural disease comprises formation of protein aggregates which are pathogenic to neurons which method comprises administering to said mammal an amount of GGA which will inhibit further pathogenic protein aggregation provided that said pathogenic protein aggregation is not related to SBMA.

### DETAILED DESCRIPTION

It is to be understood that this invention is not limited to particular embodiments described. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “an excipient” includes a plurality of excipients.

### 1. DEFINITIONS

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “neuroprotective” refers to reduced toxicity of neurons as measured in vitro in assays where neurons susceptible to degradation are protected against degradation as compared to control. Neuroprotective effects may also be evaluated in vivo by counting neurons in histology sections.

The term “neuron” or “neurons” refers to all electrically excitable cells that make up the central and peripheral nervous system. The neurons may be cells within the body of an animal or cells cultured outside the body of an animal. The term “neuron” or “neurons” also refers to established or primary tissue culture cell lines that are derived from neural cells from a mammal or tissue culture cell lines that are made to differentiate into neurons. “Neuron” or “neurons” also refers to any of the above types of cells that have also been modified to express a particular protein either extrachromosomally or intrachromosomally. “Neuron” or “neurons” also refers to transformed neurons such as neuroblastoma cells and support cells within the brain such as glia.

The term “protein aggregates” refers to a collection of proteins that may be partially or entirely mis-folded. The protein aggregates may be soluble or insoluble and may be inside the cell or outside the cell in the space between cells. Protein aggregates inside the cell can be intranuclear in which they are inside the nucleus or cytoplasm in which they are in the space outside of the nucleus but still within the cell membrane. The protein aggregates described in this invention are granular protein aggregates.

As used herein, the term “protein aggregate inhibiting amount” refers to an amount of GGA that inhibits the formation of protein aggregates at least partially or entirely. Unless specified, the inhibition could be directed to protein aggregates inside the cell or outside the cell.

As used herein, the term “intranuclear” or “intranuclearly” refers to the space inside the nuclear compartment of an animal cell.

The term “cytoplasm” refers to the space outside of the nucleus but within the outer cell wall of an animal cell.

As used herein, the term “pathogenic protein aggregate” refers to protein aggregates that are associated with disease conditions. These disease conditions include but are not limited to the death of a cell or the partial or complete loss of the neuronal signaling among two or more cells. Pathogenic protein aggregates can be located inside of a cell, for example, pathogenic intracellular protein aggregates or outside of a cell, for example, pathogenic extracellular protein aggregates.

As used herein, the term “SBMA” refers to the disease spinal and bulbar muscular atrophy. Spinal and bulbar muscular atrophy is a disease caused by pathogenic androgen receptor protein accumulation intranuclearly.

As used herein, the term “ALS” refers to amyotrophic lateral sclerosis disease.

As used herein, the term “AD” refers to Alzheimer's disease.

The term “neurotransmitter” refers to chemicals which transmit signals from a neuron to a target cell. Examples of neurotransmitters include but are not limited to: amino acids such as glutamate, aspartate, serine, γ-aminobutyric acid, and glycine; monoamines such as dopamine, norepinephrine, epinephrine, histamine, serotonin, and melatonin; and other molecules such as acetocholine, adenosine, anandamide, and nitric oxide.

The term “synapse” refers to junctions between neurons. These junctions allow for the passage of chemical signals from one cell to another.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

As used herein, the term “treatment” or “treating” means any treatment of a disease or condition in a patient, including one or more of:
preventing or protecting against the disease or condition, that is, causing the clinical symptoms not to develop, for example, in a subject at risk of suffering from such a disease or condition, thereby substantially averting onset of the disease or condition;
inhibiting the disease or condition, that is, arresting or suppressing the development of clinical symptoms; and/or
relieving the disease or condition that is, causing the regression of clinical symptoms.

The term “axon” refers to projections of neurons that conduct signals to other cells through synapses. The term “axon growth” refers to the extension of the axon projection via the growth cone at the tip of the axon.

The term “neural disease” refers to diseases that compromise the cell viability of neurons. Neural diseases in which the etiology of said neural disease comprises formation of protein aggregates which are pathogenic to neurons provided that the protein aggregates are not related to the disease SBMA and are not intranuclear, include but are not limited to ALS, AD, Parkinson's Disease, multiple sclerosis, and prion diseases such as Kuru, Creutzfeltdt-Jakob disease, Fatal familial insomnia, and Gerstmann-Straussler-Scheinker syndrome. These neural diseases are also different from SBMA in that they do not contain polyglutamine repeats. Neural diseases can be recapitulated in vitro in tissue culture cells. For example, AD can be modeled in vitro by adding pre-aggregated β-amyloid peptide to the cells. ALS can be modeled by depleting an ALS disease-related protein, TDP-43. Neural disease can also be modeled in vitro by creating protein aggregates through providing toxic stress to the cell. One way this can be achieved is by mixing dopamine with neurons such as neuroblastoma cells. These neural diseases can also be recapitulated in vivo in mouse models. A transgenic mouse that expresses a mutant Sod1 protein has similar pathology to humans with ALS. Similarly, a transgenic mouse that overexpresses APP has similar pathology to humans with AD.

The term “alkyl” refers to substituted or unsubstituted, straight chain or branched alkyl groups with C₁-C₁₂, C₁-C₆ and preferably C₁-C₄ carbon atoms.

The term “aryl” refers to a 6 to 10 membered, preferably 6 membered aryl group. An aryl group may be substituted with 1-5, preferably 1-3, halo, alkyl, and/or —O-alkyl groups.

An effective amount of GGA is the amount of GGA required to produce a protective effect in vitro or in vivo. In some embodiments the effective amount in vitro is about from 0.1 nM to about 1 mM. In some embodiments the effective amount in vitro is from about 0.1 nM to about 0.5 nM or from about 0.5 nM to about 1.0 nM or from about 1.0 nM to about 5.0 nM or from about 5.0 nM to about 10 nM or from about 10 nM to about 50 nM or from about 50 nM to about 100 nM or from about 100 nM to about 500 nM or from about 500 nM to about 1 mM. In some embodiments, the effective amount for an effect in vivo is about 0.1 mg to about 100 mg, or preferably, from about 1 mg to about 50 mg, or more preferably, from about 1 mg to about 25 mg per kg/day. In some other embodiments, the effective amount in vivo is from about 10 mg/kg/day to about 100 mg/kg/day, about 20 mg/kg/day to about 90 mg/kg/day, about 30 mg/kg/day to about 80 mg/kg/day, about 40 mg/kg/day to about 70 mg/kg/day, or about 50 mg/kg/day to about 60 mg/kg/day. In still some other embodiments, the effective amount in vivo is from about 100 mg/kg/day to about 1000 mg/kg/day.

Routes of administration refers to the method for administering GGA to a mammal. Administration can be achieved by a variety of methods. These include but are not limited to subcutaneous, intravenous, transdermal, sublingual, or intraperitoneal injection or oral administration.

The term “about” when used before a numerical designation, e.g., temperature, time, amount, and concentration, including range, indicates approximations which may vary by (+) or (−) 10%, 5%, or 1%.

The term “halogenating” is defined as converting a hydroxy group to a halo group. The term “halo” or “halo group” refers to fluoro, chloro, bromo and iodo.

The term “stereoselectively” is defined as providing over 90% of the E isomer for the newly formed double bond.

“Geometrical isomer”” or “geometrical isomers” refer to compounds that differ in the geometry of one or more olefinic centers. “E” or “(E)” refers to the trans orientation and “Z” or “(Z)” refers to the cis orientation.

Geranylgeranyl acetone (GGA) refers to a compound of the formula:

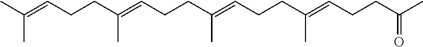

wherein compositions comprising the compound are mixtures of geometrical isomers of the compound.

The 5-trans isomer of geranylgeranyl acetone refers to a compound of the formula I:

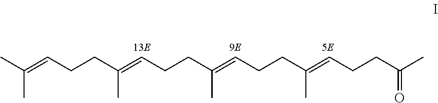

wherein the number 5 carbon atom is in the 5-trans (5E) configuration.

The 5-cis isomer of geranylgeranyl acetone refers to a compound of the formula II:

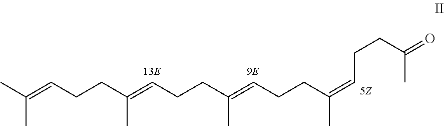

wherein the number 5 carbon atom is in the 5-cis (5Z) configuration.

### 2. COMPOUNDS

This invention relates to compounds and pharmaceutical compositions of isomers of geranylgeranyl acetone. In certain aspects, this invention relates to a synthetic 5-trans isomer compound of formula I:

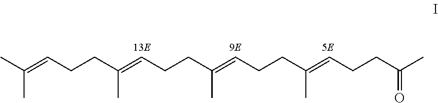

wherein I is at least 80% in the 5E, 9E, 13E configuration. In some embodiments, the invention provides for a compound of formula I wherein I is at least 85%, or at least 90%, or at least 95%, or at least 96%, or at least 97%, or at least 98%, or at least 99%, or at least 99.5%, or at least 99.9% in the 5E, 9E, 13E configuration. In some embodiments the invention for the compound of formula I does not contain any of the cis-isomer of GGA.

Another aspect of this invention relates to a synthetic 5-cis isomer compound of formula II:

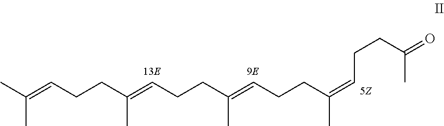

wherein II is at least 75% in the 5Z, 9E, 13E configuration. In certain embodiments, the invention provides for a compound of formula II wherein II is at least 80% in the 5E, 9E, 13E configuration, or alternatively, at least 85%, or at least 90%, or at least 95%, or at least 96%, or at least 97%, or at least 98%, or at least 99%, or at least 99.5%, or at least 99.9% in the 5E, 9E, 13E configuration. In some embodiments of the invention, the compound of formula II does not contain any of the trans-isomer of GGA.

The configuration of compounds can be determined by methods known to those skilled in the art such as chiroptical spectroscopy and nuclear magnetic resonance spectroscopy.

The data contained in the examples herewith demonstrate at low concentrations the trans-isomer of GGA is pharmacologically active and shows a dose-dependent relationship. In contrast, the cis-isomer of GGA does not demonstrate a dose dependent relationship and is deemed to be at best of minimal activity.

### 3. PHARMACEUTICAL COMPOSITIONS

In another aspect, this invention is also directed to pharmaceutical compositions comprising at least one pharmaceutically acceptable excipient and an effective amount of the trans-isomer compound of GGA according to this invention.

Pharmaceutical compositions can be formulated for different routes of administration. Although compositions suitable for oral delivery will probably be used most frequently, other routes that may be used include intravenous, intraarterial, pulmonary, rectal, nasal, vaginal, lingual, intramuscular, intraperitoneal, intracutaneous, transdermal, intracranial, and subcutaneous routes. Other dosage forms include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16^{th} ed., A. Oslo editor, Easton Pa. 1980).

The compositions are comprised of in general, GGA or a trans-isomer compound of GGA or a mixture thereof in combination with at least one pharmaceutically acceptable excipient. Acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc.

The concentration of the excipient is one that can readily be determined to be effective by those skilled in the art, and can vary depending on the particular excipient used. The total concentration of the excipients in the solution can be from about 0.001% to about 90% or from about 0.001% to about 10%.

In certain embodiments of this invention, there is provided a pharmaceutical composition comprising the compound of formula I and α-tocopherol. A related embodiment provides for a pharmaceutical composition comprising the compound of formula I, α-tocopherol, and hydroxypropyl cellulose. In another embodiment, there is provided a pharmaceutical composition comprising the compound of formula I, α-tocopherol, and gum arabic. In a further embodiment, there is a pharmaceutical composition comprising the compound of formula I, and gum arabic. In a related embodiment, there is provided the compound of formula I, gum arabic and hydroxypropyl cellulose.

When α-tocopherol is used alone or in combination with other excipients, the concentration by weight can be from about 0.001% to about 1% or from about 0.001% to about 0.005%, or from about 0.005% to about 0.01%, or from about 0.01% to about 0.015%, or from about 0.015% to about 0.03%, or from about 0.03% to about 0.05%, or from about 0.05% to about 0.07%, or from about 0.07% to about 0.1%, or from about 0.1% to about 0.15%, or from about 0.15% to about 0.3%, or from about 0.3% to about 0.5%, or from about 0.5% to about 1% by weight. In some embodiments, the concentration of α-tocopherol is about 0.001% by weight, or alternatively about 0.005%, or about 0.008%, or about 0.01%, or about 0.02%, or about 0.03%, or about 0.04%, or about 0.05% by weight.

When hydroxypropyl cellulose is used alone or in combination with other excipients, the concentration by weight can be from about 0.1% to about 30% or from about 1% to about 20%, or from about 1% to about 5%, or from about 1% to about 10%, or from about 2% to about 4%, or from about 5% to about 10%, or from about 10% to about 15%, or from about 15% to about 20%, or from about 20% to about 25%, or from about 25% to about 30% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

When gum arabic is used alone or in combination with other excipients, the concentration by weight can be from about 0.5% to about 50% or from about 1% to about 20%, or from about 1% to about 10%, or from about 3% to about 6%, or from about 5% to about 10%, or from about 4% to about 6% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

The concentration of GGA, or the trans-geranylgeranyl acetone isomer can be from about 1 to about 99% by weight in the pharmaceutical compositions provided herein. In other embodiments, the concentration of the trans-geranylgeranyl acetone isomer can be from about 1 to about 75%, or alternatively, from about 1 to about 40%, or alternatively, from about 1 to about 30%, or alternatively, from about 1 to about 25%, or alternatively, from about 1 to about 20%, or alternatively, from about 2 to about 20%, or alternatively, from about 1 to about 10%, or alternatively, from about 10 to about 20%, or alternatively, from about 10 to about 15% by weight in the pharmaceutical composition. In certain embodiments, the concentration of geranylgeranyl acetone in the pharmaceutical composition is about 5% by weight, or alternatively, about 10%, or about 20%, or about 1%, or about 2%, or about 3%, or about 4%, or about 6%, or about 7%, or about 8%, or about 9%, or about 11%, or about 12%, or about 14%, or about 16%, or about 18%, or about 22%, or about 25%, or about 26%, or about 28%, or about 30%, or about 32%, or about 34%, or about 36%, or about 38%, or about 40%, or about 42%, or about 44%, or about 46%, or about 48%, or about 50%, or about 52%, or about 54%, or about 56%, or about 58%, or about 60%, or about 64%, or about 68%, or about 72%, or about 76%, or about 80% by weight.

In one embodiment, this invention provides sustained release formulations such as drug depots or patches comprising an effective amount of GGA. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000. The patch contains, in various embodiments, an amount of GGA, preferably the 5E, 9E, 13E isomer of it, which is sufficient to maintain a therapeutically effective amount GGA in the plasma for about 12 hours. In one embodiment, the GGA comprises at least 80%, at least 85%, at least 90%, at least 95%, or at least 99% of the 5E, 9E, 13E isomer of GGA.

Compounds and pharmaceutical compositions of this invention maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In some embodiments, a compound of this invention can be used as an adjunct to conventional drug therapy.

### 4. TREATMENT METHODS

This invention provides methods for using GGA, preferably trans-GGA, still more preferably synthetic trans-GGA, or an isomer of each thereof for inhibiting neural death and increasing neural activity. For example, and without limitation, the invention provides methods for impeding the progression of neurodegenerative diseases or injury using the compound geranylgeranyl acetone (GGA). The pharmaceutical compositions and/or compounds described above are useful in the methods described herein.

In one aspect, there are methods for increasing the axon growth of neurons by contacting said neurons with an effective amount of GGA. Neural diseases can result in an impairment of signaling between neurons. This can in part be due to a reduction in the growth of axonal projections. Contacting neurons with GGA enhances axonal growth. It is contemplated that GGA will restore axonal grown in neurons afflicted with a neural disease. In a related embodiment, the pre-contacted neurons exhibit a reduction in the axon growth ability. In yet another embodiment, the GGA is the 5-trans isomer of GGA.

Methods include the use of GGA and the 5-trans isomer of GGA. In certain aspects, the 5-trans isomer of GGA has been shown to be more efficacious than the mixture of GGA, which contains both the 5-trans and 5-cis isomeric forms of GGA. Without being limited to a particular theory, it is believed that the 5-cis isomer of GGA has inhibitory properties. These inhibitory properties of the 5-cis isomer of GGA result in an attenuation of the effects exerted by the isomeric mixture and compositions of 5-trans GGA.

One embodiment of this invention is directed to a method for inhibiting the cell death of neurons susceptible to neuronal cell death, which method comprises contacting said neurons with an effective amount of GGA. Neurons susceptible to neuronal cell death include those that have the characteristics of a neurodegenerative disease and/or those that have undergone injury or toxic stress. One method of creating toxic stress to a cell is by mixing dopamine with neurons such as neuroblastoma cells. Another source of toxic stress is oxidative stress. Oxidative stress can occur from neuronal disease or injury. It is contemplated that contacting neurons with GGA will inhibit their death as measured by a MTT assay or other techniques commonly known to one skilled in the art.

In another aspect, there are methods for increasing the neurite growth of neurons by contacting said neurons with an effective amount of GGA. The term “neurite” refers to both axons and dendrites. Neural diseases can result in an impairment of signaling between neurons. This can in part be due to a reduction in the growth of axonal and/or dendritic projections. It is contemplated that contacting neurons with GGA will enhance neurite growth. It is further contemplated that GGA will restore neurite grown in neurons afflicted with a neural disease. In a related embodiment, the pre-contacted neurons exhibit a reduction in the neurite growth ability. In yet another embodiment, the GGA is the 5-trans isomer of GGA.

One embodiment of this invention is directed to a method for increasing the expression and/or release of one or more neurotransmitters from a neuron by contacting said neurons with an effective amount of GGA. It is contemplated that contacting neurons with an effective amount of GGA will increase the expression level of one or more neurotransmitters. It is also contemplated that contacting neurons with GGA will increase the release of one or more neurotransmitters from neurons. The release of one or more neurotransmitters refers to the exocytotic process by which secretory vesicles containing one or more neurotransmitters are fused to cell membrane, which directs the neurotransmitters out of the neuron. It is contemplated that the increase in the expression and/or release of neurotransmitters will lead to enhanced signaling in neurons, in which levels of expression or release of neurotransmitters are otherwise reduced due to the disease. The increase in their expression and release can be measured by molecular techniques commonly known to one skilled in the art.

One embodiment of this invention is directed to a method for inducing synapse formation of a neuron by contacting said neurons with an effective amount of GGA. A synapse is a junction between two neurons. Synapses are essential to neural function and permit transmission of signals from one neuron to the next. Thus, an increase in the neural synapses will lead to an increase in the signaling between two or more neurons. It is contemplated that contacting the neurons with an effective amount of GGA will increase synapse formation in neurons that otherwise experience reduced synapse formation as a result of neural disease.

Another embodiment of this invention is directed to a method for increasing electrical excitability of a neuron by contacting said neurons with an effective amount of GGA. Electrical excitation is one mode of communication among two or more neurons. It is contemplated that contacting neurons with an effective amount of GGA will increase the electrical excitability of neurons in which electrical excitability and other modes of neural communication are otherwise impaired due to neural disease. Electrical excitability can be measured by electrophysiological methods commonly known to one skilled in the art.

In each of the three previous paragraphs above, the administration of GGA enhances communication between neurons and accordingly provides for a method of inhibiting the loss of cognitive abilities in a mammal that is at risk of dementia or suffering from incipient or partial dementia while retaining some cognitive skills. Incipient or partial dementia in a mammal is one in which the mammal still exhibits some cognitive skills, but the skills are being lost and/or diminished over time. Method comprises administering an effective amount of GGA to said patient.

In another embodiment, this invention is directed to a method for inhibiting the death of neurons due to formation of or further formation of pathogenic protein aggregates between, outside or inside neurons, wherein said method comprises contacting said neurons at risk of developing said pathogenic protein aggregates with an amount of GGA inhibitory to protein aggregate formation, provided that said pathogenic protein aggregates are not related to SBMA. In one embodiment of this invention, the pathogenic protein aggregates form between or outside of the neurons. In another embodiment of this invention, the pathogenic protein aggregates form inside said neurons. In one embodiment of this invention, the pathogenic protein aggregates are a result of toxic stress to the cell. One method of creating toxic stress to a cell is by mixing dopamine with neurons such as neuroblastoma cells. It is contemplated that contacting neurons with GGA will inhibit their death as measured by a MTT assay or other techniques commonly known to one skilled in the art.

Another embodiment of the invention is directed to a method for protecting neurons from pathogenic extracellular protein aggregates which method comprises contacting said neurons and/or said pathogenic protein aggregates with an amount of GGA that inhibits further pathogenic protein aggregation. In one embodiment of this invention, contacting said neurons and/or said pathogenic protein aggregates with an effective amount of GGA alters the pathogenic protein aggregates into a non-pathogenic form. Without being limited to any theory, it is contemplated that contacting the neurons and/or the pathogenic protein aggregates with GGA will solubilize at least a portion of the pathogenic protein aggregates residing between, outside, or inside of the cells. It is further contemplated that contacting the neurons and/or the pathogenic protein aggregates with GGA will alter the pathogenic protein aggregates in such a way that they are non-pathogenic. A non-pathogenic form of the protein aggregate is one that does not contribute to the death or loss of functionality of the neuron. There are many assays known to one skilled in the art for measuring the protection of neurons either in cell culture or in a mammal. One example is a measure of increased cell viability by a MTT assay. Another example is by immunostaining neurons in vitro or in vivo for cell death-indicating molecules such as, for example, caspases or propidium iodide.

In yet another embodiment of the invention is directed to a method for protecting neurons from pathogenic intracellular protein aggregates which method comprises contacting said neurons with an amount of GGA which will inhibit further pathogenic protein aggregation provided that said protein aggregation is not related to SBMA. This method is not intended to inhibit or reduce, negative effects of neural diseases in which the pathogenic protein aggregates are intranuclear or diseases in which the protein aggregation is related to SBMA. SBMA is a disease caused by pathogenic androgen receptor protein accumulation. It is distinct from the neural diseases mentioned in this application since the pathogenic protein aggregates of SBMA contain polyglutamines and are formed intranuclearly. It is also distinct from the neural diseases described in this application because the protein aggregates are formed from androgen receptor protein accumulation. It is contemplated that contacting neurons with an effective amount of GGA will alter the pathogenic protein aggregate into a non-pathogenic form.

One embodiment of the invention is directed to a method of modulating the activity of G proteins in neurons which method comprises contacting said neurons with an effective amount of GGA. It is contemplated that contacting neurons with GGA will alter the sub-cellular localization, thus changing the activities of the G protein in the cell. In one embodiment of the invention, contacting neurons with GGA will enhance the activity of G proteins in neurons. It is contemplated that contacting GGA with neurons will increase the expression level of G proteins. It is also contemplated that contacting GGA with neurons will enhance the activity of G proteins by changing their sub-cellular localization to the cell membranes where they must be to exert their biological activities.

One embodiment of the invention is directed to a method of modulating or enhancing the activity of G proteins in neurons at risk of death which method comprises contacting said neurons with an effective amount of GGA. Neurons may be at risk of death as a result of genetic changes related to ALS. One such genetic mutation is a depletion of the TDP-43 protein. It is contemplated that neurons with depleted TDP-43 or other genetic mutations associated with ALS will have an increase or change in the activity of G proteins after being contacted with GGA. It is further contemplated that GGA will result in an increase in the activity of G proteins in these cells by changing their sub-cellular localization to the cell membranes where they must be to exert their biological activities.

Another embodiment of the invention is directed to a method for inhibiting the neurotoxicity of β-amyloid peptide by contacting the β-amyloid peptide with an effective amount of GGA. In one embodiment of the invention the β-amyloid peptide is between or outside of neurons. In yet another embodiment of the invention, the β-amyloid peptide is part of the β-amyloid plaque. It is contemplated that contacting neurons with GGA will result in solubilizing at least a portion of the β-amyloid peptide, thus decreasing its neurotoxicity. It is further contemplated that GGA will decrease the toxicity of the β-amyloid peptide by altering it in such a way that it is no longer toxic to the cell. It is also believed that GGA will induce the expression of heat shock proteins (HSPs) in the neurons. It is also contemplated that HSPs will be induced in support cells such as glial cells. The induced heat shock proteins in the neurons or glial cells may be transmitted extracellularly and act to dissolve extracellular protein aggregates. Cell viability can be measured by standard assays known to those skilled in the art. One such example of an assay to measure cell viability is a MTT assay. Another example is a MTS assay. The modulation of protein aggregation can be visualized by immunostaining or histological staining techniques commonly known to one skilled in the art.

One embodiment of the invention is directed to a method for inhibiting neural death and increasing neural activity in a mammal suffering from neural diseases, wherein the etiology of said neural diseases comprises formation of protein aggregates which are pathogenic to neurons, and which method comprises administering to said mammal an amount of GGA which will inhibit further pathogenic protein aggregation. This method is not intended to inhibit neural death and increase neural activity in neural diseases in which the pathogenic protein aggregates are intranuclear or diseases in which the protein aggregation is related to SBMA.

Neural diseases such as AD and ALS disease have the common characteristic of protein aggregates either inside neural cells in cytoplasm or in the extracellular space between two or more neural cells. This invention relates to a method for using the compound GGA to inhibit the formation of the protein aggregates or alter the pathogenic protein aggregates into a non-pathogenic form. It is contemplated that this will attenuate some of the symptoms associated with these neural diseases.

In one embodiment the mammal is a human afflicted with a neural disease. In one embodiment of this invention, the negative effect of the neural disease being inhibited or reduced is ALS. ALS is characterized by a loss of functionality of motor neurons. This results in the inability to control muscle movements. ALS is a neurodegenerative disease that does not typically show intranuclear protein aggregates. It is contemplated that GGA will prevent or inhibit the formation of extracellular or intracellular protein aggregates that are cytoplasm, not intranuclear and not related to SBMA. It is also contemplated that GGA will alter the pathogenic protein aggregates into a form that is non-pathogenic. Methods for diagnosing ALS are commonly known to those skilled in the art. Additionally, there are numerous patents that describe methods for diagnosing ALS. These include U.S. Pat. No. 5,851,783 and U.S. Pat. No. 7,356,521 both of which are incorporated herein by reference in their entirety.

In one embodiment of the invention the negative effect of the neural disease being inhibited or reduced is AD. AD is a neurodegenerative disease that does not typically show intranuclear protein aggregates. It is contemplated that GGA will prevent or inhibit the formation of extracellular or intracellular protein aggregates. It is also contemplated that GGA will alter the pathogenic protein aggregates into a form that is non-pathogenic. Methods for diagnosing AD are commonly known to those skilled in the art. Additionally, there are numerous patents that describe methods for diagnosing AD. These include U.S. Pat. No. 6,130,048 and U.S. Pat. No. 6,391,553 both of which are incorporated herein by reference in their entirety.

In another embodiment, the mammal is a laboratory research mammal such as a mouse. In one embodiment of this invention, the neural disease is ALS. One such mouse model for ALS is a transgenic mouse with a Sod1 mutant gene. It is contemplated that GGA will enhance the motor skills and body weights when administered to a mouse with a mutant Sod1 gene. It is further contemplated that administering GGA to this mouse will increase the survival rate of Sod1 mutant mice. Motor skills can be measured by standard techniques known to one skilled in the art. In yet another embodiment of this invention, the neural disease is AD. One example of a transgenic mouse model for AD is a mouse that overexpresses the APP (Amyloid beta Precursor Protein). It is contemplated that administering GGA to a transgenic AD mouse will improve the learning and memory skills of said mouse. It is further contemplated that GGA will decrease the amount and/or size of β-amyloid peptide and/or plaque found inside, between, or outside of neurons. The β-amyloid peptide or plaque can be visualized in histology sections by immunostaining or other staining techniques.

In one embodiment of the invention administering GGA to a mammal alters the pathogenic protein aggregate present into a non-pathogenic form. In another embodiment of the invention, administering GGA to a mammal will prevent pathogenic protein aggregates from forming.

Another aspect of this invention relates to a method for reducing seizures in a mammal in need thereof, which method comprises administering a therapeutically effective amount of GGA, thereby reducing seizures. The reduction of seizures refers to reducing the occurrence and/or severity of seizures. In one embodiment, the seizure is epileptic seizure. In another embodiment, the methods of this invention prevent neural death during epileptic seizures. The severity of the seizure can be measured by one skilled in the art.

In methods described herein, the GGA refers to the compounds and/or pharmaceutical compositions described previously of the cis isomer, the trans isomer or the mixture of GGA. In such methods, it is contemplated that the trans isomer may exhibit a more efficacious result compared to the mixture or the cis isomer. It is also contemplated that the inhibitory effects of the cis isomer allow it to be used to attenuate the effects of the mixture or the trans isomer in the above-described methods. Therefore, in one embodiment of each method, the GGA used is the trans isomer of GGA. In another embodiment, the GGA used is the cis isomer of GGA. In yet another embodiment, the method comprises contacting the neuron with an effective amount of the 5-cis isomer to attenuate the effect of the mixture or 5-trans isomer.

In certain aspects, the methods described herein relate to administering GGA or the isomeric compounds or compositions of GGA in vitro. In other aspects the administration is in vivo. In yet other aspects, the in vivo administration is to a mammal. Mammals include but are not limited to humans and common laboratory research animals such as, for example, mice, rats, dogs, pigs, cats, and rabbits.

### 5. SYNTHETIC METHODS

This invention provides a synthetic method comprising one or more of the following

steps:

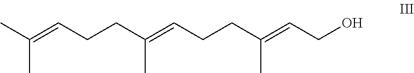

(i) reacting a compound of formula III under halogenation conditions to provide a compound of formula IV;

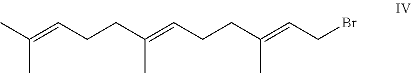

(ii) reacting the compound of formula IV with alkyl acetoacetate under alkylation conditions to provide a compound of formula V, where the stereochemistry at sterogenic center can be a racemic, R or S configuration:

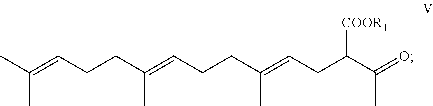

(iii) reacting the compound of formula V under hydrolysis and decarboxylation conditions to provide a compound of formula VI:

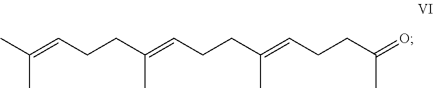

(iv) reacting the compound of formula VI with a compound of formula VII:

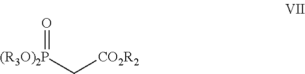

wherein R₂ and each R₃ independently are alkyl or substituted or unsubstituted aryl, under olefination conditions to selectively provide a compound of formula VIII:

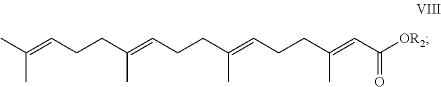

(v) reacting the compound of formula VIII under reduction conditions to provide a compound of formula IX

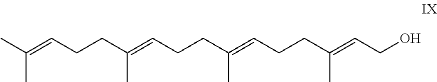

Compound III is combined with at least an equimolar amount of a halogenating agent typically in an inert solvent. As used in this application, an “inert solvent” is a solvent that does not react under the reaction conditions in which it is employed as a solvent. The reaction is typically run at a temperature of about 0° C. to 20° C. for a period of time sufficient to effect substantial completion of the reaction. Suitable solvents include, by way of example only, diethyl ether, acetonitrile, and the like. Suitable halogenating agents include PBr₃ or PPh₃/CBr₄. After reaction completion, the resulting product, compound IV, can be recovered under conventional conditions such as extraction, precipitation, filtration, chromatography, and the like or, alternatively, used in the next step of the reaction without purification and/or isolation.

Compound IV is combined with at least an equimolar amount of an alkyl acetoacetate, in the presence of a base and an inert solvent. The reaction is typically run initially at 0° C., and then warmed up to room temperature for a period of time sufficient to effect substantial completion of the reaction. Suitable solvents include, by way of example only, various alcohols, such as ethanol, dioxane, and mixtures thereof. Suitable bases include, by way of example only, alkali metal alkoxides, such as sodium ethoxide.

Compound V is reacted with at least an equimolar amount, preferably, an excess of aqueous alkali. The reaction is typically run at about 40 to 80° C. and preferably about 80° C. for a period of time sufficient to effect substantial completion of the reaction. Suitable solvents include, by way of examples only, alcohols, such as methanol, ethanol, and the like.

Compound VI is combined with at least an equimolar amount, preferably, an excess of a compound of formula VII, and at least an equimolar amount, preferably, an excess of base, in an inert solvent. The reaction is typically run, initially at about −30° C. for about 1-2 hours, and at room temperature for a period of time sufficient to effect substantial completion of the reaction. Suitable solvents include, by way of examples only tetrahydrofuran, dioxane, and the like. Suitable bases include, by way of example only, alkali metal hydrides, such as sodium hydride, or potassium hexamethyldisilazide (KHMDS), or potassium tertiary butoxide (^{t}BuOK).

Compound VIII is combined with a reducing agent in an inert solvent. The reaction is typically run at about 0° C. for about 15 minutes, and at room temperature for a period of time sufficient to effect substantial completion of the reaction. Suitable reducing agents include, without limitation, LiAlH₄. Suitable solvents include, by way of examples only diethyl ether, tetrahydrofuran, dioxane, and the like.

AS will be apparent to the skilled artisan, after reaction completion, the resulting product, can be recovered under conventional conditions such as precipitation, filtration, chromatography, and the like or, alternatively, used in the next step of the reaction without purification and/or isolation.

In some embodiments, the method further comprises repeating steps (i), (ii), and (iii) sequentially with compound of formula VIII to provide the compound of formula I, wherein m is 2.

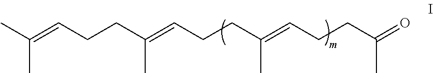

In another embodiment, the method or procedure further comprises repeating steps (i), (ii), (iii), (iv), and (v), sequentially, 1-3 times.

In another of its synthetic method aspects, there is provided a method comprising one or more of the following steps:

(i) reacting a compound of formula IIIB:

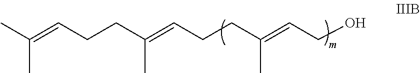

wherein m is 1-3, under halogenation conditions to provide a compound of formula IVB:

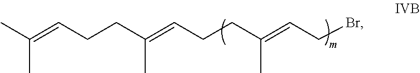

(ii) reacting the compound of formula IVB with alkyl acetoacetates, under alkylating conditions to provide a compound of formula VB, where the stereochemistry at sterogenic center can be a racemic, R or S configuration:

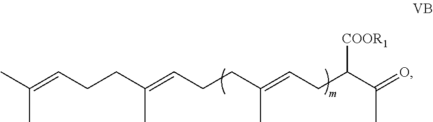

wherein R¹ alkyl is substituted or unsubstituted alkyl
(iii) reacting a compound of formula VB under hydrolysis and decarboxylation conditions to provide a compound of formula VIB:

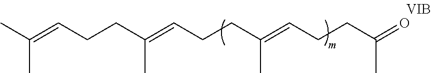

In another of its synthetic method aspects, this invention provides a method comprising step (i) or step (ii) or steps (i)+(ii):

(i) reacting a compound of formula XC:

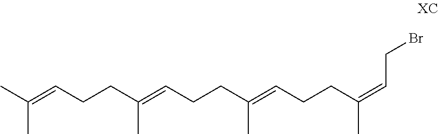

with alkyl acetoacetate under alkylating conditions to provide a compound of formula XIC, the stereochemistry at sterogenic center can be a racemic, R or S configuration:

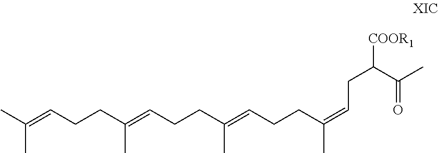

wherein R1 is as defined herein, and
(ii) reacting the compound XIC obtained under hydrolysis and decarboxylation conditions to provide a compound of formula II:

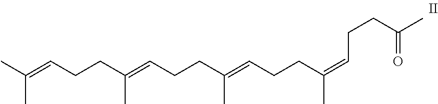

As will be apparent to the skilled artisan, the various reaction steps leading to compound VIB or to the 5Z isomer are performed in the manner described hereinabove.

In another of its synthetic method aspects, this invention provides a method comprising reacting a ketal compound of formula XII:

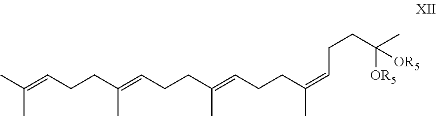

wherein each R₅ independently is C₁-C₆ alkyl, or two R₅ groups together with the oxygen atoms they are attached to form a 5 or 6 membered ring, which ring is optionally substituted with 1-3, preferably 1-2, C₁-C₆ alkyl groups, under hydrolysis conditions to provide a compound of formula II.

The ketal is combined with at least a catalytic amount, such as, 1-20 mole % of an aqueous acid, preferably, an aqueous mineral acid in an inert solvent. The reaction is typically run about 25° C. to about 80° C., for a period of time sufficient to effect substantial completion of the reaction. Suitable acids include, without limitation, HCl, H₂SO₄, and the like. Suitable solvents include alcohols, such as methanol, ethanol, tetrahydrofuran, and the like.

In another embodiment, this invention provides a method comprising reacting a compound of formula XI:

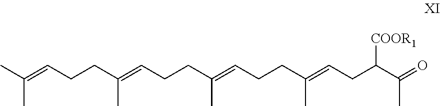

under hydrolysis and subsequently decarboxylation conditions to form a compound of formula I:

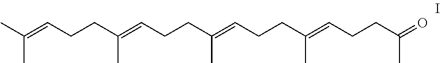

Alternatively, reacting compound of formula VII with X followed by in situ hydrolysis and decarboxylation of compound with formula XI can afford the compound of formula I.

In another embodiment, this invention provides a method comprising reacting a compound of formula XIC:

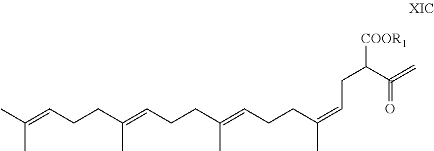

under hydrolysis and subsequent decarboxylation conditions to form the compound of formula II

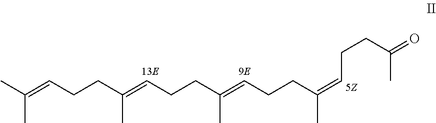

Hydrolysis and decarboxylation conditions useful in these methods will be apparent to the skilled artisan upon reading this disclosure.

It will also be apparent to the skilled artisan that the methods further employ routine steps of separation or purification to isolate the compounds, following methods such as chromatography, distillation, or crystallization.

### 6. UTILITY

GGA is a known anti-ulcer drug used commercially and in clinical situations. GGA has also been shown to exert cytoprotective effects on a variety of organs, such as the eye, brain, and heart (See for example Ishii Y., et al., Invest Ophthalmol V is Sci 2003; 44:1982-92; Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 104:1837-43; and Suzuki S, et al., Kidney Int 2005; 67:2210-20).

In certain situations, the concentration of GGA required to exert a cytoprotective effect is an excessive amount of more than 600 mg per kg per day (Katsuno et al., Proc. Natl. Acad. Sci. USA 2003, 100, 2409-2414). The trans-isomer of GGA has been shown to be more efficacious at lower concentrations than a composition containing from 1:2 to 1:3 cis:trans mixture of GGA, and a composition of the cis-isomer of GGA alone. Therefore, the trans-isomer of GGA is useful for exerting cytoprotective effects on cells at a lower concentration than the cis-isomer or the 1:2 to 1:3 mixture of cis and trans isomers. Surprisingly, increasing amounts of the cis-isomer was found to antagonize the activity of the trans-isomer, as exemplified below.

It is contemplated that the isomeric mixture of GGA and/or compositions containing the 5-trans isomer of GGA can be used to inhibit neural death and increase neural activity in a mammal suffering from a neural disease, wherein the etiology of said neural disease comprises formation of protein aggregates which are pathogenic to neurons which method comprises administering to said mammal an amount of GGA which will inhibit neural death and increase neural activity, or impede the progression of the neural disease. As it relates to the isomeric mixture of GGA, this method is not intended to inhibit or reduce the negative effect of a neural disease in which the pathogenic protein aggregates are intranuclear or diseases in which the protein aggregation is related to SBMA.

Negative effects of neural diseases that are inhibited or reduced by GGA and the 5-trans isomer of GGA according to this invention include but are not limited to Alzheimer's disease, Parkinson's disease, multiple sclerosis, prion diseases such as Kuru, Creutzfeltdt-Jakob disease, Fatal familial insomnia, and Gerstmann-Straussler-Scheinker syndrome, amyotrophic lateral sclerosis, or damage to the spinal cord. GGA and the 5-trans isomer of GGA are also contemplated to prevent neural death during epileptic seizure.

### 7. ASSAYS

The isolated cis- and trans-compounds described herein are also useful in assays which access a compound having putative cytoprotective effects. In particular, in such assays, the cis-isomer of GGA will behave as baseline or negative control and the trans-isomer as a positive control. The putative compound is tested in the assay described in Example 10 and its activity correlated against the cis- and trans-isomers. Compounds exhibiting activity similar to or exceeding that of the trans-isomer would be considered to be active compounds. Compounds providing activity similar to the cis-isomer would be considered to be inactive compounds. Accordingly, the cis-isomer finds utility as a negative control in the assay.

### 8. EXAMPLES OF THE INVENTION

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

In the examples below as well as throughout the application, the following abbreviations have the following meanings If not defined, the terms have their generally accepted meanings
° C.=degrees Celsius
PBr₃=phosphorus tribromide
EE=ethyl ether
EtOH=Ethanol
NaOEt=sodium ethoxide
Oet=Ethoxide
N=Normal
KOH=potassium hydroxide
aq=aqueous
h=hour(s)
RT=Room temperature
LAH=lithium aluminum hydride
THF=Tetrahydrofuran
min=minute(s)
Et=Ethyl
MeOH=Methanol
NaH=sodium hydride
ON=Overnight
E or (E)=Trans
Z or (Z)=Cis
TLC=thin layer chromatography
GGA=geranylgeranyl acetone
μL=Microliter
mL=Milliliter
PK=negative logarithm of the dissociation constant K
HPC=hydroxypropyl cellulose
DI=Deionized
Mn=number average molar mass
Av=Average
p-TsOH=p-toluenesulfonic acid
Ph₃P=Triphenylphosphine
Br-=bromide ion
CBr₄=Tetrabromomethane
LC-MS=Liquid chromatography—mass spectrometry
Rf=retardation factor
PEG-200 polyethylene glycol
KHMDA=potassium hexamethylenediamine
ACN=Acetonitrile
TBDMS=tert-butyldimethyl silyl
Kp=Ratio Of AUC_{brian} to AUCₚₗₐₛₘₐ
AUC=Area Under the curve

LC-MS Parameters for Analysis

### System: Agilent 1100 LC-MSD

### Parameters:

Sample Concentration: 7.2 mg in 1.44 mL DMSO (5 mg/mL). Dilute 10 uL to 0.5
mL acetonitrile (100 ug/mL)
HPLC Column: Xterra MS, C18, 50×2.1, 3.5 micron

### Column Temperature: 40° C.

Mobile Phase A: 0.1% formic acid in water
Mobile Phase B: 0.1% formic acid in acetonitrile
Flow Rate: 0.3 mL/min

### Injection Volume: 5 uL

### Gradient LC-MS:

| time (min) | B (%) |
|---|---|
| 0 | 5 |
| 15 | 100 |
| 25 | 100 |
| 25.1 | 5 |
| 30 | 5 |

### MS Parameters:

### Ion Source Electrospray

### Polarity: Positive

### Mass Range: 100-1000 amu

### Fragmentor: 80

Dry Gas: 10 l/min
Dry Gas temp: 350° C.

### Vcap: 4000

### Nebulizer Pressure: 35

### Gain: 5

The starting materials for the reactions described below are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1 15 (John Wiley and Sons, 1991), Rodd's Chemistry of Carbon Compounds, Volumes 1 5 and Supplementals (Elsevier Science Publishers, 1989), Organic Reactions, Volumes 1 40 (John Wiley and Sons, 1991), March's Advanced Organic Chemistry, (John Wiley and Sons, 4.sup.th Edition), and Larock's Comprehensive Organic Transformations (VCH Publishers Inc., 1989).

### Example 1

### 5E,9E,13E-Geranylgeranyl Acetone Synthesis

Synthesis of 5-trans-Isomer: 5E,9E,13E-Geranylgeranyl acetone 1: The synthesis of 5-trans isomer: 5E,9E,13E-geranylgeranyl acetone 1 can be achieved as per outlined in the scheme-1.

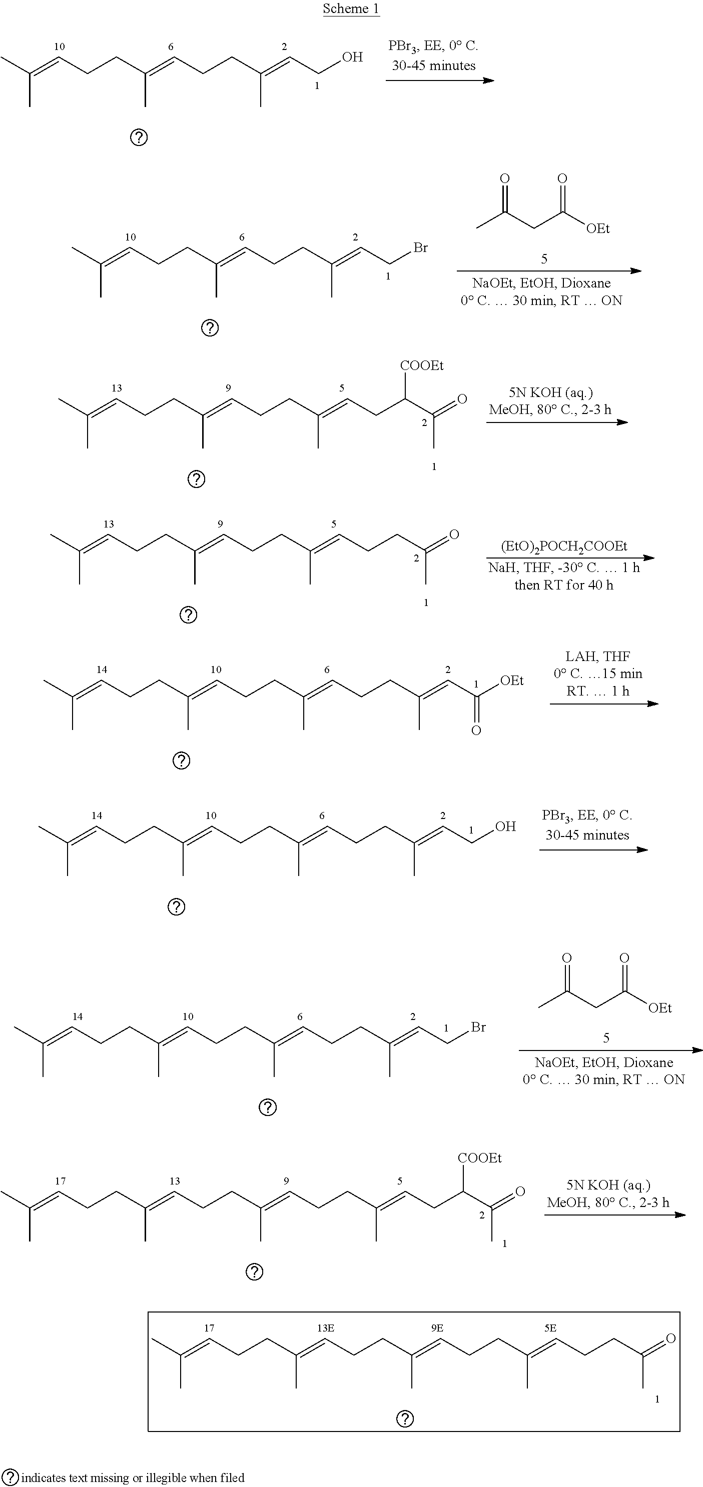

The 2E,6E-farnesyl alcohol 3 (where the geometry at C2 and C6 positions is already fixed as trans- or E) was designed and used as a commercially available starting material for the synthesis of 5E,9E,13E-geranylgeranyl acetone 1. The alcohol function of 2E, 6E-farnesyl alcohol 3 was converted to the corresponding bromide 4 by the treatment of phosphorus tribromide (PBr₃) in ethyl ether (EE) or with Ph₃P and CBr₄ in acetonitrile (ACN) at 0° C. The resulting bromide was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7. A one pot conversion of bromide 4 to the corresponding farnesyl acetone 7 can be possible without isolating intermediate ketoester 6.

In order to generate the trans-orientation of olefin at C2 of conjugated olefin 8 in a key step, the reaction of 5E,9E-farnesyl acetone 7 with carbanion [derived from the reaction of (EtO)₂PO—CH₂—COOEt and sodium hydride (NaH)] at −30° C. was conducted to obtain the desired 2E,6E,10E-conjugated ester 8. The formation of the product 8 with the exclusive trans (E) geometry was observed when the reaction was conducted at −30° C. or temperature below −30° C., where all the three olefins are set in a trans (E) orientation (Ref.: Kato et al., J. Org. Chem. 1980, 45, 1126-1130 and Wiemer et al., Organic Letters, 2005, 7(22), 4803-4806). The minor cis-(Z)-isomer was eliminated/separated from the trans-(E)-isomer 8 by a careful silica gel column chromatographic purification. However, it was also noted that the formation the corresponding cis-isomer (Z) was increased when the reaction was conducted at 0° C. or at higher temperature. It was also noted that the mixture of cis (2Z)- and trans (2E)-isomer of 8 can be separated by a very careful column chromatographic separation.

The resulting 2E-conjugated ester 8 was reduced to the corresponding 2E-alcohol 9 by means of a lithium aluminum hydride (LAH) treatment, which was then converted into the corresponding 2E,6E,10E-geranylgeranyl bromide 10 by means of phosphorus tribromide (PBr₃) treatment in ethyl ether (EE) or with Ph₃P and CBr₄ in acetonitrile (ACN) at 0° C. Furthermore, the interaction of carbanion (derived from ethyl acetoacetate 5 and sodium ethoxide) with the bromide 10 at 0° C. afforded the desired 2E,6E,10E-geranylgeranyl ketoester 11, a precursor needed for 5E,9E,13E-geranylgeranyl acetone 1. The subsequent ester hydrolysis and decarboxylation of ketoester 11 using aq. 5N KOH at 80° C. yielded the requisite 5E,9E,13E-geranylgeranyl acetone 1. TLC Rf: 0.28 (5% Ethyl Acetate in Hexanes); LC Retention time: 16.68 min; MS (m/e): 313 [M−18+H]⁺, 331 [MH]+, 353 [M+K].

### Example 2

### 5-Z,9E,13E-Geranylgeranyl Acetone Synthesis

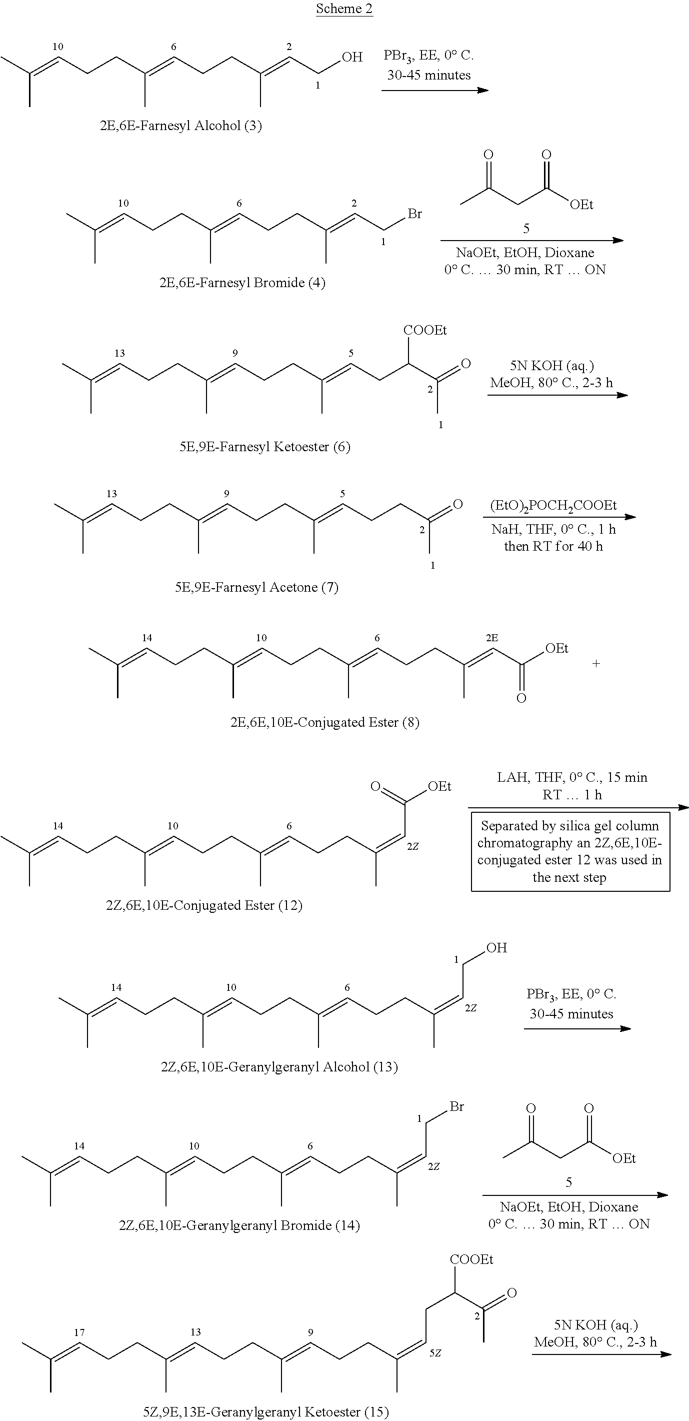

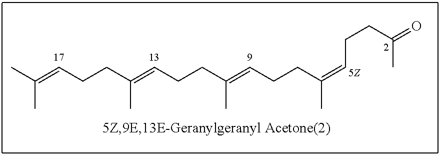

The 2E,6E-farnesyl alcohol 3 (where the geometry at C2 and C6 positions is already fixed as trans- or E) was used as a commercially available starting material for the synthesis of 5Z,9E,13E-geranylgeranyl acetone 2. The reaction of farnesyl alcohol 3 with phosphorus tribromide (PBr₃) in ethyl ether (EE) or with Ph₃P and CBr₄ in acetonitrile (ACN) at 0° C. afforded the requisite bromide 4, which was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7, one of the key intermediate for the synthesis of 5E,9E,13E-geranylgeranyl acetone 1 and 5Z,9E,13E-geranylgeranyl acetone 2.

With a view to obtain product with cis-geometry at C2 with the conjugated olefin 12, the reaction of 5E,9E-farnesyl acetone 7 with carbanion [derived from the reaction of (EtOH)₂PO—CH₂—COOEt and sodium hydride (NaH)] at 0° C. was conducted. This reaction afforded a mixture of 2E,6E,10E-conjugated ester 8 and 2Z,6E,10E-conjugated ester 12, from which the C2-cis (Z)-isomer 12 was separated by a repeated and careful silica gel column chromatography (Ref. Kato et al., J. Org. Chem., 1980, 45, 1126-1130).

The resulting 2Z-conjugated ester 12 was converted into the corresponding 2Z-alcohol 13 by means of a lithium aluminum hydride (LAH) treatment. The 2Z-alcohol 13 was transformed into the corresponding 2Z,6E,10E-geranylgeranyl bromide 14 by using phosphorus tribromide (PBr₃) treatment in ethyl ether (EE) or with Ph₃P and CBr₄ acetonitrile (ACN) at 0° C., and then reacted with carbanion (derived from ethyl acetoacetate 5 and sodium ethoxide) at 0° C. afforded the desired 2Z,6E,10E-geranylgeranyl ketoester 15, a precursor needed for 5Z,9E,13E-geranylgeranyl acetone 2. The subsequent ester hydrolysis and decarboxylation of ketoester 15 using aq. 5N KOH at 80° C. yielded the requisite 5Z,9E,13E-geranylgeranyl acetone 2.

### Example 3

### 5Z,9E,13E-Geranylgeranyl Acetone Synthesis

Alternative synthesis of 5-cis Isomer: 5Z,9E,13E-Geranylgeranyl acetone 2: The alternative synthesis of 5Z,9E,13E-geranylgeranyl acetone 2 can be achieved as shown in the scheme-3.

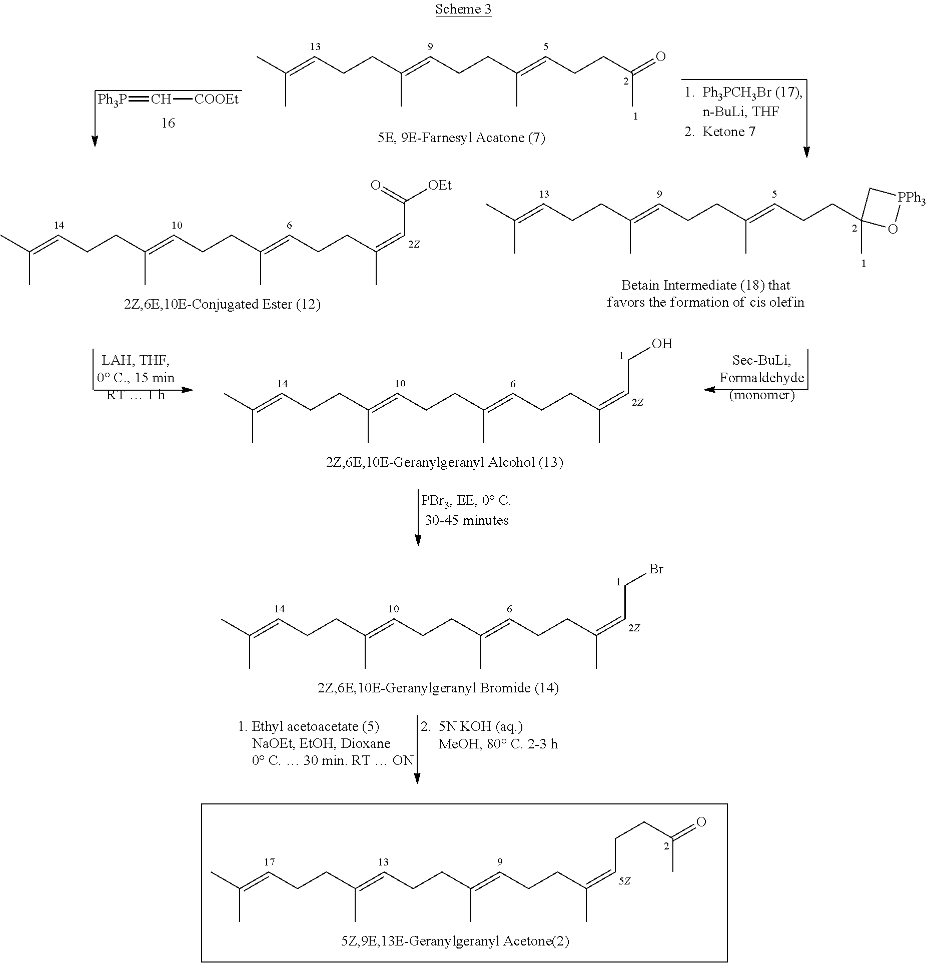

The use of 5E,9E-farnesyl acetone 7, as a key intermediate, can be used to generate additional double bond with cis-(Z)-orientation. In one approach, the reaction of 5E,9E-farnesyl acetone 7 with the witting reagent 16 can afford the conjugated ester 12 with cis-(Z)-geometry at C2 position. The subsequent reduction of ester 12 with lithium aluminum hydride (LAH) can generate the corresponding alcohol 13, which then can be converted into the corresponding bromide 14. The conversion of bromide 14 to the ketoester 15 followed by hydrolysis and decarboxylation can afford the desired 5-cis (Z) isomer; 5Z,9E,13E-geranygeranyl acetone (2).

In an alternative approach, the reaction of 5E,9E-farnesyl acetone 7 with triphenyl methylphosphonrane bromide 17 under a basic conditions followed by treatment with formaldehyde (monomeric) can afford the 2Z,6E10E-geranylgeranyl alcohol 13 with cis (Z)-orientation at C2 (Ref.: Wiemer et al., Organic Letters, 2005, 7(22), 4803-4806). The conversion of bromide 14 to the ketoester 15 followed by hydrolysis and decarboxylation can afford the desired 5-cis (Z)-isomer; 5Z,9E,13E-geranygeranyl acetone (2). TLC Rf: 0.32 (5% Ethyl Acetate in Hexanes); LC: Retention time: 17.18 min; MS (m/e): 313 [M−18+H]+, 331 [MH, very weak ionization]+, 339 [M−CH₂+Na], 353[M+K].

All the intermediate products were purified by silica gel column chromatography and then used in the next step, except the bromides 4, 10 and 14. Due to the unstable nature of bromides 4, 10 and 14 towards silica gel column chromatography, these bromides were used in the next step without purification. Alternatively, all the intermediate products shown in the schemes 1, 2 and 3 are liquids and therefore can be separated and purified by a distillation process under appropriate levels of vacuum. All the intermediates and final products were characterized by LC-MS for mass along with the Thin Layer Chromatography (TLC) for Rf values.

### Example 4

### 5-Z,9E,13E-Geranylgeranyl Acetone Synthesis

Alternative synthesis of 5-cis Isomer: 5Z,9E,13E-Geranylgeranyl acetone 2: The alternative synthesis of 5Z,9E,13E-geranylgeranyl acetone 2 can be achieved as shown in the scheme-4

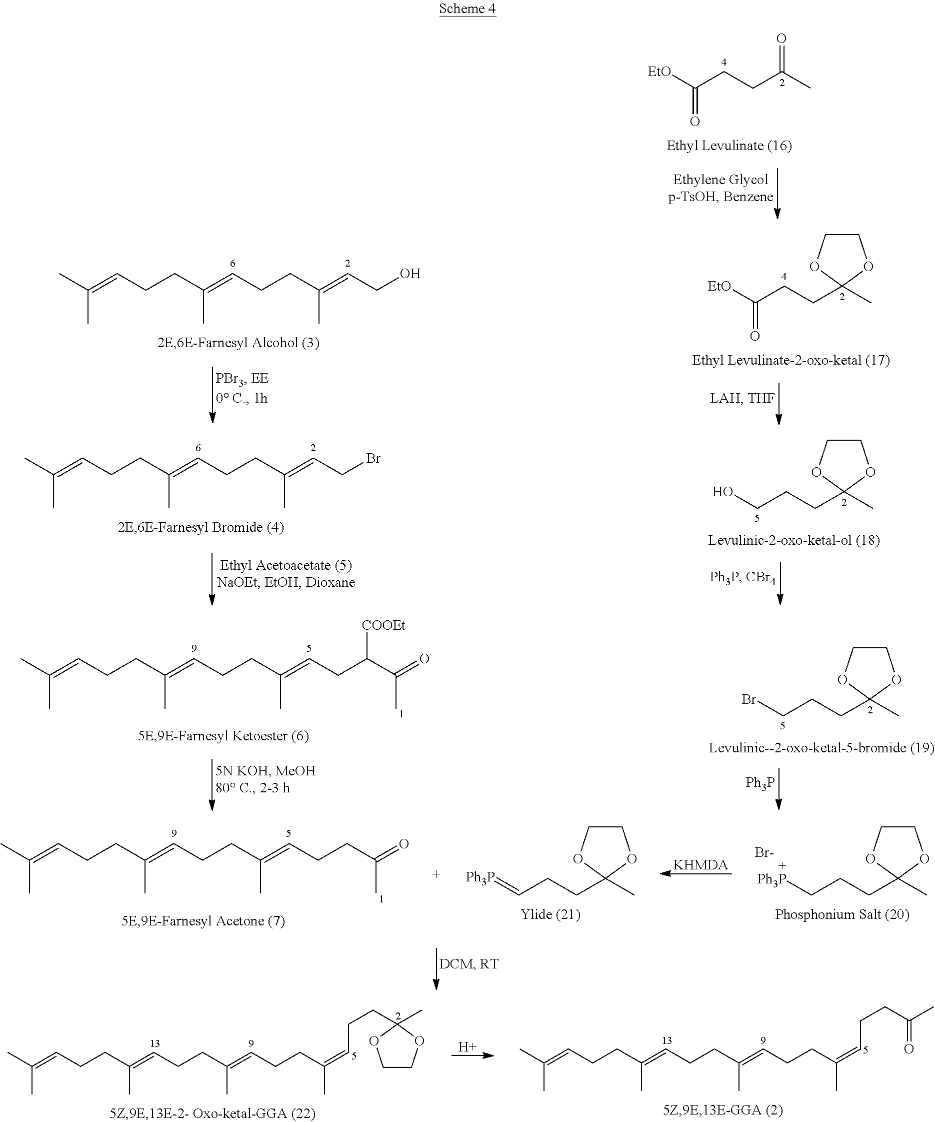

The convergent synthesis of 5Z,9E,13E-GGA 2 has been shown in the above scheme and is outlined as follows.

The 2E,6E-farnesyl alcohol 3 (where the geometry at C2 and C6 positions is already fixed as trans- or E) was used as a commercially available starting material for the synthesis of 5Z,9E,13E-geranylgeranyl acetone 2. The reaction of farnesyl alcohol 3 with phosphorus tribromide (PBr₃) in ethyl ether (EE) or with Ph₃P and CBr₄ in acetonitrile (ACN) at 0° C. afforded the requisite bromide 4, which was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7, one of the key intermediate for the synthesis of 5E,9E,13E-geranylgeranyl acetone 1 and 5Z,9E,13E-geranylgeranyl acetone 2.

The other synthon, namely the ylide 21 can be synthesized from a commercially available starting material, ethyl levulinate 16, a sugar industry by-product. The ketalization of ethyl levulinate 16 using conventional conditions (ethylene glycol, p-TsOH, azeotropic reflux) can yield the desired 2-oxo-ketal 17, which then can be reduced using LAH in THF at 0° C. to the corresponding alcohol 18. Furthermore, the alcohol 18 then can be treated with Ph₃Br in diethyl ether at 0° C. to obtain the bromide 19, which then after treatment with Ph₃P can yield the phosphonium bromide salt 20. The bromide salt 20 upon treatment with mild alkali (1N NaOH) can furnish the desired ylide 21, required to complete the synthesis of 5Z-GGA 2.

With a view to obtain product with cis-geometry, the reaction of 5E,9E-farnesyl acetone 7 with the ylide 21 in DCM at RT can afford the desired 5Z-oxoketal 22 (Ref: Ernest et al, Tetrahedron Lett. 1982, 23(2), 167-170). The protected oxo-function from 22 can be removed by means of a mild acid treatment to yield the expected 5Z,9E,13E-GGA 2.

### Example 5

### 5E,9E,13E-Geranylgeranyl Acetone Synthesis

Alternative synthesis of 5-trans Isomer: 5E,9E,13E-Geranylgeranyl acetone 1: The alternative synthesis of 5E,9E,13E-geranylgeranyl acetone 1 can be achieved as shown in the scheme-5.

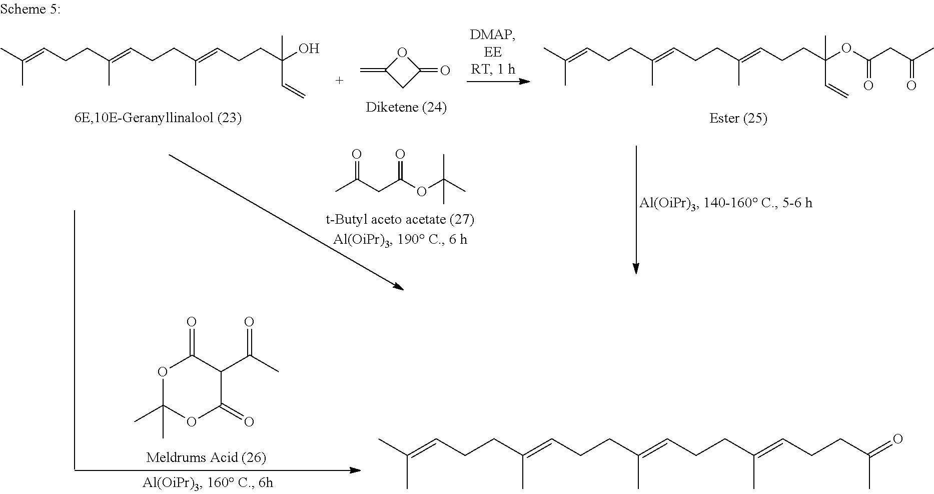

The 5E, 9E, 13E-geranyl geranyl acetone (1) can be prepared by reacting 6E-10E-geranyl linalool (23) with diketene (24) catalyzed by DMAP in ethyl ether to give the ester 25. The ester 25 in the Carroll rearrangement using Al(OiPr)₃ at elevated temperature can afford the desired 5E, 9E, 13E-geranyl geranyl acetone (1). In another approach, the GGA (1) can be prepared by treating geranyl linalool (23) with the Meldrum's acid 26 in the Carroll rearrangement using Al(OiPr)₃ at 160° C. Similarly, the use of tert-butyl acetoacetate (27) with geranyl linalool (23) in the Carroll rearrangement can also give the desired 5E, 9E, 13E-geranyl geranyl acetone (1).

### Example 6

### 5-Z,9E,13E-Geranylgeranyl Acetone Synthesis

The alternative synthesis of 5Z,9E,13E-geranylgeranyl acetone 2 can be achieved as shown in the scheme-6.

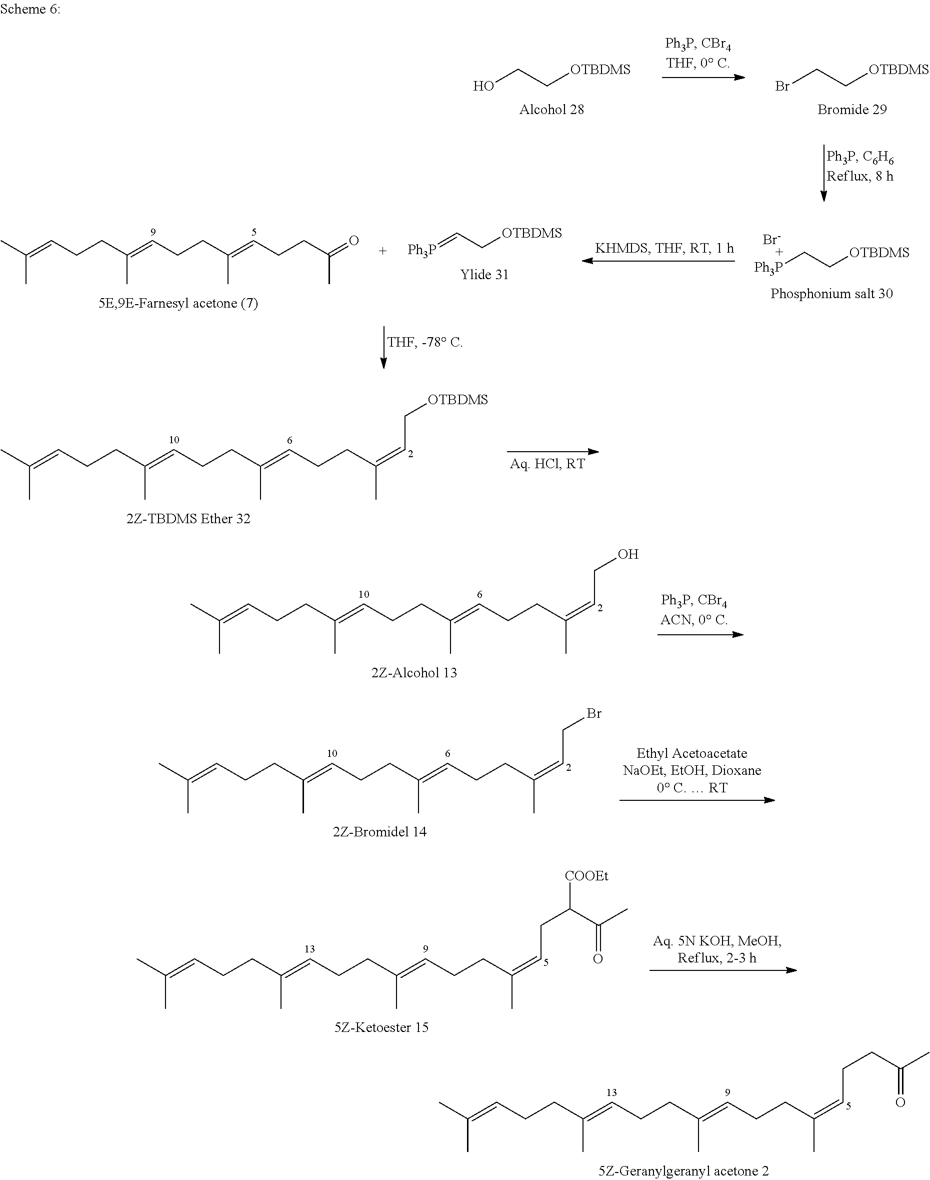

Alternative synthesis of 5-cis Isomer: 5Z,9E,13E-Geranylgeranyl acetone 2: The 2E,6E-farnesyl alcohol 3 (where the geometry at C2 and C6 positions is already fixed as trans- or E) was used as a commercially available starting material for the synthesis of 5Z,9E,13E-geranylgeranyl acetone 2. The reaction of farnesyl alcohol 3 with phosphorus tribromide (PBr₃) in ethyl ether (EE) or with Ph₃P and CBr₄ in acetonitrile (ACN) at 0° C. afforded the requisite bromide 4, which was then reacted with carbanion (derived from the reaction of ethyl acetoacetate 5 and sodium ethoxide) to yield the desired 5E,9E-farnesyl ketoester 6. The homologated ketoester 6 after hydrolysis and decarboxylation using aqueous 5N KOH yielded the expected 5E,9E-farnesyl acetone 7, one of the key intermediate for the synthesis of 5E,9E,13E-geranylgeranyl acetone 1 and 5Z,9E,13E-geranylgeranyl acetone 2.

The ylide 31 synthesized from a commercially available mono-TBDMS protected ethylene glycol 28. The conversion of alcohol function of 28 by using Ph₃P and CBr₄ in acetonitrile can afford the corresponding bromide 29, which then can be used to make a phosphonium bromide salt 30 by treatment with Ph₃P at elevated temperature. The bromide salt 30 upon treatment with KHMDS in THF can afford the ylide 31, which then can be reacted in-situ with ketone 7 in a key step to establish cis geometry with the newly created double bond at C2 position and obtain the 2Z-TBDMS ether 32 (ref: Still et al, J. Org. Chem., 1980, 45, 4260-4262 and Donetti et al, Tetrahedron Lett. 1982, 23(21), 2219-2222). The deprotection of TBDMS with aqueous HCl to afford the corresponding alcohol 13 followed by conversion of alcohol to bromide using Ph₃P and CBr₄ can afford the desired bromide 14. The bromide 14 upon reaction with ethyl acetoacetate can give ketoester 15, which then upon hydrolysis followed by decarboxylation can yield the desired 5-Z-GGA (5-cis) 2.

### Formulations and Pharmacokinetics (PK) Studies

With a view to administer the geranylgeranyl acetone (GGA) effectively to determine its PK and efficacy, preclinical research formulations, exemplified in Example 6-20, have been developed. The use of isomeric mixture of 5E-and 5Z-geranylgeranyl acetone (referred as GGA) was employed during the development of preclinical research formulations. It is contemplated that synthesized compositions of 5E- and/or 5Z-GGA can be used in such preclinical research formulations.

In the following examples, Plasma concentrations and PK parameters were obtained from CNS-101 IV dosing and oral formulation PK studies. PK PARAMETERS were calculated from noncompartmental analysis (NCA) model using WinNonlin software and the linear/log trapezoidal method.

Definitions for PK parameters:

Parameters that do not require 1z: Tₘₐₓ (min): Time to reach Cmax (directly taken from analytical data).

Parameters that requires 1z: Terminal Half-Life (t_{1/2})=ln(2)/1z. Calculated using Lambda_z method to find best fit. If necessary, the concentration-time points were manually selected for use in the calculation. Bolded-italicized concentrations indicate points used for calculation.

### Bioavailability

$$F ( \% ) = Bioavailability = \frac{AUC ( PO ) / Dose ( PO )}{AUC ( PO ) / Dose ( PO )} \times 100$$

### Example 6

### GGA Formulation using 5% Gum Arabic with 0.008% α-tocopherol

TABLE 1
Using 5% Gum Arabic with 0.008% α-tocopherol

| Entry | Gum Arabic (μL) | GGA (μL) | GGA (%)* |
|---|---|---|---|
| 1. | 190 μL | 10 μL | 5% |
| 2. | 180 μL | 20 μL | 10% |
| 3. | 160 μL | 40 μL | 20% |
| 4. | 140 μL | 60 μL | 30% |

*The % ratios are based on volumes

Preparation of 5% Gum Arabic Solution: 1.25 g of Gum Arabic was suspended in DI water (23.75 mL; until the total volume was 25 mL) and agitated using agitator until all Gum Arabic was miscible in DI water. To this solution was added α—tocopherol (2 μL, final concentration=0.008) and agitated to obtain a solution of 5% gum Arabic, which was then used as a stock solution to formulate GGA.

Preparation of GGA suspension in 5% Gum Arabic Aqueous Solution: To a respective amount of 5% of Gum Arabic solution from the stock, the corresponding amount of GGA was added and the resulting mixture was agitated to obtain an aqueous suspension formulation.

The in-vivo PK studies by using rat species with GGA in 5% Gum Arabic as an aqueous suspension formulation resulted in 37.3% oral bioavailability (% F) with t1/2=3.43 h and Tmax=7.33 h. The in-vivo studies to obtain Kp, which is a ratio of AUC_{brain} to AUCₚₗₐₛₘₐ was done in rat species at 6 h and 8 h time points and found that the GGA has Kp from 0.08 to 0.11.

### Example 7

### GGA Formulation using Hydroxypropyl Cellulose (HPC; Av. Mn=100,000; High Average Molecular Weight) with 0.008% α-tocopherol

TABLE 2
Hydroxypropyl Cellulose (HPC; Av. Mn = 100,000; High Average
Molecular Weight) with 0.008% α-tocopherol

| Entry | 3% HPC (μL) | GGA (μL) | GGA (%)* |
|---|---|---|---|
| 1. | 475 μL | 25 μL | 5% |
| 2. | 450 μL | 50 μL | 10% |
| 3. | 425 μL | 75 μL | 15% |
| 4. | 400 μL | 100 μL | 20% |

*The % ratios are based on volumes

Preparation of 3% Hydroxypropyl Cellulose Solution: To a mixture of 3 g of hydroxypropyl cellulose (Av. Mn=100,000) and α-tocopherol (8 μL, final concentration=0.008%) was added DI water (˜97 mL) until the total volume reached 100 mL. The resulting mixture was agitated to obtain a stock solution to formulate the GGA.

Preparation of GGA suspension in 3% Hydroxypropyl Cellulose Aqueous Solution: To a respective amount of 3% of hydroxypropyl cellulose solution from the stock, the corresponding amount of GGA was added and the resulting mixture was agitated to obtain an aqueous suspension formulation.

The in-vivo PK studies by using rat species with GGA in 3% Hydroxypropyl Cellulose (HPC, Av, Mn=100,000) as an aqueous suspension formulation resulted in 41.8% oral bioavailability (% F) with t1/2=3.13 h and Tₘₐₓ=8.66 h.

### Example 8

### GGA Formulation using Hydroxypropyl Cellulose (HPC; Av. Mn=10,262; Low Average Molecular Weight) with 0.008% α-tocopherol

TABLE 3
Hydroxypropyl Cellulose (HPC; Av. Mn = 10,262; Low Average
Molecular Weight) with 0.008% α-tocopherol

| Entry | 3% HPC (μL) | GGA (μL) | GGA (%)* |
|---|---|---|---|
| 1. | 475 μL | 25 μL | 5% |
| 2. | 450 μL | 50 μL | 10% |
| 3. | 425 μL | 75 μL | 15% |
| 4. | 400 μL | 100 μL | 20% |

*The % ratios are based on volumes

Preparation of 3% Hydroxypropyl Cellulose Solution: To a mixture of 3 g of hydroxypropyl cellulose (Av. Mn=10,262) and α-tocopherol (8 μL, final concentration=0.008%) was added DI water (˜97 mL) until the total volume reached 100 mL. The resulting mixture was agitated to obtain a stock solution to formulate the GGA.

Preparation of GGA suspension/solution in 3% Hydroxypropyl Cellulose Solution: To a respective amount of 3% of hydroxypropyl cellulose solution from the stock, the corresponding amount of GGA was added and the resulting mixture was agitated to obtain an aqueous suspension formulation.

The in-vivo PK studies by using rat species with GGA in 3% Hydroxypropyl Cellulose (HPC, Av, Mn=10,262) as an aqueous suspension formulation resulted in 35% oral bioavailability (% F) with t1/2=18.73 h and Tmax=9.33 h.

### Example 9

### GGA Formulation using 5% Gum Arabic+3% Hydroxypropyl Cellulose (HPC; Av. Mn=100,000; High Average Molecular Weight) and with 0.008% α-tocopherol

TABLE 4
5% Gum Arabic + 3% Hydroxypropyl Cellulose (HPC; Av.
Mn = 100,000; High Average Molecular Weight) and with 0.008%
α-tocopherol

| | 5% Gum | | | |
|---|---|---|---|---|
| | Arabic (0.008% | | | |
| | α-tocopherol) | | | GGA |
| Entry | (μL) | 3% HPC | GGA (μL) | (%)* |
| 1. | 460 μL | 15 mg | 25 μL | 5% |
| 2. | 450 μL | 15 mg | 50 μL | 10% |
| 3. | 410 μL | 15 mg | 75 μL | 15% |
| 4. | 385 μL | 15 mg | 100 μL | 20% |

*The % ratios are based on volumes

A. Preparation of 5% Gum Arabic Solution: 1.25 g of Gum Arabic was suspended in DI water (23.75 mL; until the total volume was 25 mL) and agitated using agitator until all gum Arabic was miscible in DI water. To this solution was added α-tocopherol (2 μL, 0.008%) and agitated for a minute to obtain 5% gum Arabic

Preparation of GGA suspension/solution in 5% Gum Arabic+3% Hydroxypropyl Cellulose (Av. Mn=100,000): To a respective amount of 5% of Gum Arabic solution from the stock, the corresponding amount of GGA and hydroxypropyl cellulose (Av. Mn=100,000) were added and the resulting mixture was agitated to obtain an aqueous suspension formulation.

The in-vivo PK studies by using rat species with GGA in 5% Gum Arabic+3% Hydroxypropyl Cellulose (Av. Mn=100,000) as an aqueous suspension formulation resulted in 58% oral bioavailability (% F) with t1/2=10.2 h and Tmax=5.33 h.

### Example 10

### GGA Formulation using 5% Gum Arabic+3% Hydroxypropyl Cellulose (HPC; Av. Mn=10,262; Low Av. Molecular Weight) and with 0.008% α-tocopherol

TABLE 5
5% Gum Arabic + 3% Hydroxypropyl Cellulose (HPC; Av.
Mn = 10,262; Low Av. Molecular Weight) and with 0.008% α-tocopherol

| | 5% Gum | | | |
|---|---|---|---|---|
| | Arabic (0.008% | | | |
| | α-tocopherol) | | | GGA |
| Entry | (μL) | 3% HPC | GGA (μL) | (%)* |
| 1. | 460 μL | 15 mg | 25 μL | 5% |
| 2. | 450 μL | 15 mg | 50 μL | 10% |
| 3. | 410 μL | 15 mg | 75 μL | 15% |
| 4. | 385 μL | 15 mg | 100 μL | 20% |

*The % ratios are based on volumes

A. Preparation of 5% Gum Arabic Solution: 1.25 g of Gum Arabic was suspended in DI water (23.75 mL; until the total volume was 25 mL) and agitated until all gum Arabic was miscible in DI water. To this solution was added α-tocopherol (2 μL, final concentration=0.008%) and agitated for a minute to obtain a solution of 5% gum Arabic, which was then used as a stock solution to formulate GGA.

Preparation of GGA suspension/solution in 5% Gum Arabic+3% Hydroxypropyl Cellulose (Av. Mn=100,000): To a respective amount of 5% of Gum Arabic solution from the stock, the corresponding amount of GGA and hydroxypropyl cellulose (Av. Mn=10,262) were added and the resulting mixture was agitated to afford an aqueous suspension.

The in-vivo PK studies by using rat species with GGA in 5% Gum Arabic+3% Hydroxypropyl Cellulose (Av. Mn=10, 262) as an aqueous suspension formulation resulted in 36.5% oral bioavailability (% F) with t1/2=6.73 h and Tmax=13.3 h.

### Example 11

### Culturing of Primary Motor Neurons from Rats

Rat primary motor neurons were isolated from embryonic spinal cords in accordance with the method of Henderson et al.; J Cohen and G P Wilkin (ed.), Neural Cell Culture, (1995) p69-81 which is herein incorporated by reference in its entirety. Briefly, spinal cords were dissected from day 15 embryo (E15) and incubated in a trypsin solution, and followed by DNase treatment to release spinal cord cells from tissue fragments. The cell suspension was centrifuged to remove tissue fragments. Then motor neurons were enriched by density gradient centrifugation.

Motor neurons were cultured in serum-free neurobasal medium containing insulin, forskolin, 3-isobutyl-1-methylxanthine, neurotrophic factors, Bovine serum albumin, selenium, transferrin, putrescine, progesterone and B27 supplement in tissue culture plate coated with poly-ornithine and laminin.

### Example 12

### 5-Trans isomer of GGA (CNS-102) is More Efficacious In Vitro than the Isomer Mixture of GGA (CNS-101)

Rat primary motor neurons were prepared and cultured as described Example 11. Various concentration of CNS-101, which is a mixture of 5-trans and 5-cis isomer (cis:trans ratio=1:2-1:3). CNS-102 (herein also referred to as 5-trans isomer of GGA), and CNS-103 (herein also referred to as 5-cis isomer of GGA) were added to the culture at the time of plating the cells. The cells extending axons were counted in five different fields for each treatment after 72 hrs. Percentage of positive cells relative to total cells in the same magnification field was calculated and the results were expressed as means+/− standard deviations, n=5. The EC₅₀ is a measure of the effectiveness of a compound, and corresponds to the concentration at which the drug exhibits half its maximum effect. These results are depicted in the table below:

| GGA | EC₅₀ |
|---|---|
| CNS-101 | 6.1 nM (4-7 nM)* |
| CNS-102 | 0.92 nM (0.5-2.0 nM)* |
| CNS-103 | 9.49 nM (8-12 nM)* |

*values in parenthesis indicate a reasonable range expected for the EC₅₀

### Example 13

### A Large Quantity of GGA Isomer Mixture (CNS-101) Inhibited Viability of Neuroblastoma Cells

Human SH-SY5Y neuroblastoma cells were culture in DMEM/HAM F12 supplemented with 10% fetal bovine serum (FBS) for 24 hrs. The cells were treated with retinoic acid in DMEM/HAM F12 medium supplemented with 5% FBS for 48 hrs. Then the cells were treated with CNS-101 (100 micro molar (μM)) or vehicle, dimethyl sulfoxide for 48 hrs. Cell viability was determined using ATP detection assay (Promega). These results are depicted in the table below:

| | Mean of cell viability | |
|---|---|---|
| GGA | (arbitrary units) | SE |
| 100 μM CNS-101 | 1181020 | 25815 |
| Vehicle | 1340600 | 23409 |

P < 0.001

### Example 14

### A Large Quantity of GGA Isomer Mixture (CNS-101) and Cis-Isomer (CNS-103) Inhibited Viability of Neuroblastoma Cells

Mouse Neuro2A neuroblastoma cells were cultured in DMEM supplemented with 10% FBS for 24 hrs. The cells were treated with various concentrations of CNS-101, CNS-102, and CNS-103 as indicated for 48 hrs. Then differentiation was induced by retinoic acid in DMEM supplemented with 2% FBS. An inhibitor against a G-protein, GGTI-298, was incubated. After 24 hrs incubation, cells with neurites were counted. A large quantity of GGA isomer mixture (CNS-101) and the cis-isomer (CNS-103) inhibited viability of neuroblastoma cells. These results are depicted in the table below:

| | Mean of cell numbers | |
|---|---|---|
| GGA | (Arbitrary units) | SE |
| CNS-101 (10 μM) | 0.551 | 0.1333 |
| CNS-102 (10 μM) | 0.738 | 0.0018 |
| CNS-103 (10 μM) | 0.195 | 0.0933 |

P < 0.03

The data in examples 13 and 14 support the conclusion that the cis isomer, CNS-103, has a deleterious effect on cell viability and that the trans isomer has a positive effect. The two examples taken together suggest that at higher concentrations, the cis isomer has an inhibitory effect on the trans isomer. Example 16 further elaborates upon these findings.

### Example 15

### Effects of the GGA Isomer Mixture (CNS-101) on Cells Experiencing Oxidative Stress

Human SH-SY5Y neuroblastoma cells were culture in DMEM/HAM F12 supplemented with 10% fetal bovine serum (FBS) for 2 days. The cells were treated with retinoic acid in DMEM/HAM F12 medium supplemented with 5% FBS for 48 hrs. Then the cells were treated with various concentrations of CNS 101 for 48 hrs. Cells were exposed to hydrogen peroxide (75 micro M) or DMEM/HAM F12(control) for 2 hrs, then cell viability was determined using ATP assay (Promega). These results are depicted in the table below:

| GGA | Mean of cell viability |
|---|---|
| CNS-101 (10 μM) | 6-10% |
| Vehicle | 3-5% |

100% of cell viability was evaluated in the absence of hydrogen peroxide and CNS-101.

### Example 16

### Effects of the GGA Isomer Mixture (CNS-101), the trans-isomer (CNS-102) and the cis-isomer (CNS-103), and an Inhibitor of a G-protein (GGTI-298), on the Viability of Cells

Neuro2A cells were cultured with CNS-101, CNS-102, or CNS-103 in the presence or absence of an inhibitor against a G-protein (GGTI-298). After differentiation was induced, cells that extended neurites were counted. These results are depicted in the table below:

| | Mean of cell numbers |
|---|---|
| GGA | (Arbitrary units) |
| CNS-101 (0.1-1 μM) | 0.45-0.65 |
| CNS-102 (0.1-1 μM) | 0.45-0.65 |
| CNS-103 (0.1-1 μM) | 0.20-0.45 |
| Vehicle | 0.0-0.20 |

### Example 17

### The GGA Isomer Mixture (CNS-101) Activated Neurite Outgrowth of Neuroblastoma Cells

Human SH-SY5Y neuroblastoma cells were cultured in DMEM/HAM F12 supplemented with 10% fetal bovine serum (FBS) for 24 hrs. The cells were treated with retinoic acid in DMEM/HAM F12 medium supplemented with 5% FBS. Then the cells were treated with various concentrations of CNS-101. Total length of neurites for each treatment was measured. These results are depicted in the table below:

| | Mean of neurite |
|---|---|
| GGA | outgrowth |
| CNS-101 (0.1 μM) | 125% |
| CNS-101 (1 μM) | 166% |
| CNS-101 (10 μM) | 194% |
| Vehicle | 100% |

Data were spread in a range of +/−10% from each mean.

### Example 18

### The GGA Isomer Mixture (CNS-101) and the trans-isomer (CNS-102) Alleviated Neurodegenerations Induced by Kainic Acid

CNS-101 or CNS-102 were orally dosed to Sprague-Dawley rats, and Kainic acid was injected. Seizure behaviors were observed and scored (Ref R.J. Racine, Modification of seizure activity by electrical stimulation: U. Motor seizure, Electroencephalogr. Neurophysiol. 32 (1972) 281-294. Modifications were made for the methods). Brain tissues of rats were sectioned on histology slides, and neurons in hippocampus tissues were stained by Nissl. Neurons in dentate gyms tissues damaged by Kainic acid were quantified. The Memantine composition used in comparison refers to a commercially available NMDA receptor agonist These results are depicted in the tables below:

| GGA | |
|---|---|
| | Hippocampus dentate |
| | gyrus neurons damaged |
| | (Arbitrary units) |
| CNS-101 | 0.725 |
| Vehicle | 20.9 |
| Memantine | 3.53 |
| P-value to vehicle data | P < 0.05 |
| | Seizure behaviors scores |
| CNS-102 | 18.8 |
| Vehicle | 34 |
| Memantine | 36.2 |
| P-value to vehicle data | P < 0.11 |

### Example 19

### Comparison of the Efficacy of CNS-101 and CNS-102 in Alleviating Neurodegeneration Induced by Kainic Acid

CNS-101, CNS-102 or a vehicle only control were orally dosed to Sprague-Dawley rats, and Kainic ainic acid was injected. Seizure behaviors were observed and scored (Ref R.J. Racine, Modification of seizure activity by electrical stimulation: 11. Motor seizure, Electroencephalogram. Clin, Neurophysiol. 32. (1972) 281-294. Modifications were made for the methods). Brain tissues of rats were sectioned on histology slides, and neurons in hippocampus tissues were stained by Nissl. Neurons damaged by Kainic acid and behavior scores were quantified.

These results indicate that a lower concentration of the trans-isomer of GGA is more efficacious at protecting neurons from neuronal damage than a higher concentration of either the isomer mixture of the cis-isomer of GGA. Furthermore, it is contemplated that such effects of trans-GGA also renders it useful for protecting tissue damage during seizures, ischemic attacks, and neural impairment such as in glaucoma.

| | Mean of Hippocampus | |
|---|---|---|
| | CA3 neurons damaged | Mean of Seizure |
| GGA | (Arbitrary units) | behaviors scores |
| CNS-102 (3 mg/Kg rat) | 10.25 | 27.5 |
| CNS-102 (12 mg/Kg rat) | 10.16 | 22.5 |
| Vehicle | 37.67 | 50.5 |
| P value to vehicle data | P < 0.085 | P < 0.165 |
| CNS-102 (25 mg/Kg rat) | 1.97 | 25.75 |
| Vehicle | 9.43 | 38 |
| P value to vehicle data | P < 0.142 | P < 0.025 |
| CNS-101 (25 mg/Kg rat) | 37.38 | 33.83 |
| Vehicle | 38.77 | 28.5 |

| | Mean of Hippocampus |
|---|---|
| | CA3 neurons damaged |
| GGA | (Arbitrary units) |
| CNS-103 (12 mg/Kg rat) | 8.16 |
| CNS-103 (25 mg/Kg rat) | 10.64 |
| Vehicle | 10.71 |

### Example 20

### GGA's Effect on the Activity of G Proteins in a Neuron

Neuroblastoma cells can be obtained from the American Type Culture Collection (ATCC) and cultured according to the suggested culturing techniques of ATCC. The cultured cells will be contacted with an effective amount of GGA. The change in G protein activity will be monitored by a western blot of lysates obtained from subcellular fractionation of cells. Subcellular fractionation can be performed using commercially available kits (from Calbiochem for example) according to the manufacturer's protocol. The western analysis will be performed using subcellular fractions from the membrane and cytoplasmic compartments of cells. The western blot will be performed according to standard molecular biology techniques using antibodies directed to the different G proteins: RHOA, RAC1, CDC42, RASD2. It is contemplated that reacting the neuroblastoma cells with an effective amount of GGA will modulate the active, membrane-bound portion of RHOA, RAC1, CDC42, and/or RASD2. Interaction of those small G-proteins with gene products involved in protein aggregations will also be tested. Those gene products include Huntington gene product (Htt), sumoylation machinery, etc.

The same assay will be performed using neuroblastoma cells or other neurons that are depleted for the TDP-43 protein. TDP-43 depleted cells mimic the effects of neurodegeneration related to ALS. TDP-43 depletion can be accomplished using the siRNA and/or shRNA technologies. It is contemplated that neurons which are susceptible to neurodegeneration by TDP-43 depletion will have a change in the G protein activity after said neurons are contacted with an effective amount of GGA. It is further contemplated that reacting said neurons with an effective amount of GGA will increase the active membrane-bound portion of the G proteins.

The same assay will be performed using neuroblastoma cells or other neurons that are susceptible to neurodegeneration due to inhibition of geranylgeranylation of the G proteins. GGTI-298 is a specific inhibitor of geranylgeranylation and increases neuronal cell death through inhibiting the activation of G proteins by geranylgeranylation. Therefore, GGTI-298 and GGA will both be contacted with tissue cultures of neuroblastoma cells. It is contemplated that neurons which are susceptible to neurodegeneration by GGTI-298 will have a change in the G protein activity after said neurons are contacted with an effective amount of GGA. It is further contemplated that reacting said neurons with an effective amount of GGA will increase the active membrane-bound portion of the G proteins.

### Example 21

### GGA's Effect on the Pathogenicity of Protein Aggregates in Neurons Susceptible to Neurodegeneration

Cultured neuroblastoma cells can be made susceptible to neurodegeneration by mixing the cells with dopamine. The addition of dopamine to the cells will cause pathogenic protein aggregates in the cytoplasm. To test the effect of GGA on neurons susceptible to neurodegeneration, an effective amount of dopamine will be first contacted with the neurons to induce pathogenic protein aggregate formation in the cells. Next, an effective amount of GGA will be contacted with said neurons. The change in the size and/or number of protein aggregates will then be measured using histological staining techniques and/or immunostaining techniques commonly known to one skilled in the art. It is contemplated that contacting GGA with neurons susceptible to neurodegeneration due to dopamine-induced protein aggregation will solubilize at least a portion of the protein aggregate, thus decrease the pathogenicity to the cell. It is further contemplated that contacting GGA with neurons susceptible to neurodegeneration due to dopamine-induced protein aggregation will alter the form of the pathogenic protein aggregate into a non-pathogenic form, thus decrease the pathogenicity to the cell.

Contacting neurons in vitro with β-amyloid peptide aggregates will recapitulate the toxic effects of AD due to β-amyloid peptide aggregates in vivo. To test if GGA reduces the pathogenicity of β-amyloid peptide aggregates in cultured neuroblastoma cells, the β-amyloid peptide aggregates will be added directly to the cell culture medium of the cultured cells. The β-amyloid peptide can be purchased commercially and aggregated in vitro. An effective amount of GGA will then be added to the cell culture to test for a modulation of the pathogenicity to the cells. It is contemplated that contacting GGA with neurons susceptible to neurodegeneration due to β-amyloid peptide aggregation will solubilize at least a portion of the protein aggregate, thus decrease the pathogenicity to the cell. It is further contemplated that contacting GGA with neurons susceptible to neurodegeneration due to β-amyloid peptide aggregation will alter the form of the pathogenic protein aggregate into a non-pathogenic form, thus decrease the pathogenicity. The change in the size and/or number of protein aggregates will then be measured using histological staining techniques and/or immunostaining techniques commonly known to one skilled in the art.

### Example 22

### GGA's Effect In Vivo in Mammals Susceptible to Neurodegeneration

Neurotoxins can be used to recapitulate the effect of AD in mice. To test the effects of administering GGA to a mammal that is susceptible to AD, neurotoxins will be administered systemically or by direct injection into the brain tissues of mice to induce the pathology associated with AD. The neurotoxins will be administered either before, simultaneously, or after the administration of GGA. The GGA may be administered to said mice mixed with a pharmaceutically acceptable excipient. These mice will then be monitored for survival rate, neuron density in brain tissues, and learning, memory, and motor skills. The learning, memory, and motor skills are measured by techniques commonly known to one skilled in the art. It is contemplated that treating the animal with an effective amount of GGA will attenuate some of the symptoms associated with the injection of the neurotoxin.

There are a variety of mouse models available that are engineered to have the same pathology associated with different human diseases. One such mouse model is a mouse that over-expresses the Amyloid beta Precursor Protein (APP). This mouse has a similar pathology to that seen in human AD. An effective amount of GGA will be administered to mice over-expressing APP. The GGA may be administered to said mice mixed with a pharmaceutically acceptable excipient. These mice will then be monitored for body weight, β-amyloid plaque formation, and learning, memory, and motor skills. Histology sections of these mice will also be analyzed by staining and immunohistochemical techniques to detect changes in the brain after GGA administration. It is contemplated that treating the animal with an effective amount of GGA will attenuate some of the symptoms associated with AD.

Mice expressing a Sod1 mutant protein exhibit similar pathology to humans with ALS. An effective amount of GGA will be administered to Sod1 mutant mice. The GGA may be administered to said mice mixed with a pharmaceutically acceptable excipient. These mice will then be monitored for survival rate, body weight, and motor skills. Histology sections of these mice will also be analyzed by histology staining and immunohistochemical techniques to detect changes in the brain, spinal cords, or muscles after GGA administration. It is contemplated that treating the Sod1 mutant mice with an effective amount of GGA will increase the survival rate, body weights, and enhance the motor skills of these mice.

From the foregoing it will be appreciated that, although specific embodiments of the invention have been described herein for purposes of illustration, various modifications may be made without deviating from the spirit and scope of the invention.

Throughout the description of this invention, reference is made to various patent applications and publications, each of which are herein incorporated by reference in their entirety.

What is claimed is:

1. A compound, which is synthetic 5E, 9E, 13E geranylgeranyl acetone.

2. The compound of claim 1, which is free of 5Z, 9E, 13E geranylgeranyl acetone II.

3. A synthetic 5-cis isomer compound of formula II: 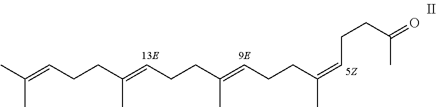
or a ketal thereof of formula XII: 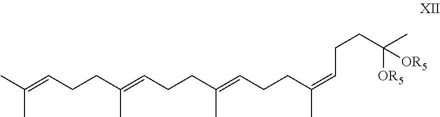
wherein each R₅ independently is C₁-C₆ alkyl, or two R₅ groups together with the oxygen atoms they are attached to form a 5 or 6 membered ring, which ring is optionally substituted with 1-3, preferably 1-2, C₁-C₆ alkyl groups.

4. A pharmaceutical composition comprising the compound of claim 1 or 2, and at least one pharmaceutical excipient. 1.

5. The pharmaceutical composition of claim 4, wherein the pharmaceutical excipient is one or more of α-tocopherol and optionally one or more of hydroxypropyl cellulose and gum Arabic.

6. A composition for increasing the expression and/or release of one or more neurotransmitters from a neuron at risk of developing pathogenic protein aggregates associated with AD or ALS, said composition comprising a protein aggregate inhibiting amount of GGA, or an isomer or a mixture of isomers thereof.

7. A composition for increasing the expression and/or release of one or more neurotransmitters from a neuron at risk of developing extracellular pathogenic protein aggregates, said composition comprising an extracellular protein aggregate inhibiting amount of GGA, or an isomer or a mixture of isomers thereof.

8. A method for:
(i) neuroprotection of neurons at risk of neural damage or death,
(ii) increasing the axon growth of a neuron,
(iii) inhibiting the cell death of a neuron susceptible to neuronal cell death,
(iv) increasing the neurite growth of a neuron, or
(v) neurostimulation comprising increasing the expression and/or the release of one or more neurotransmitters from a neuron,
the method comprising contacting said neurons with an effective amount of GGA.

9. The method of claim 8, wherein the GGA is the 5-trans isomer of GGA.

10. The method of claim 8, wherein said pre-contacted neuron exhibits one or more of:
(i) a reduction in the axon growth ability,
(ii) a reduced expression level of one or more neurotransmitters,
(iii) a reduction in the formation of synapses, and
(iv) a reduction in electrical excitability.

11. The method of claim 8, wherein the neurostimulation comprises one or more of:
(i) enhancing or inducing synapse formation of a neuron,
(ii) increasing or enhancing electrical excitability of a neuron,
(iii) modulating the activity of G proteins in neurons,
(iv) enhancing the activation of G proteins in neurons.

12. A method for inhibiting the loss of cognitive abilities in a mammal that is at risk of dementia or suffering from incipient or partial dementia while retaining some cognitive skills which method comprises contacting said neuron with an effective amount of a 5-trans isomer of GGA.

13. A method for inhibiting the death of neurons due to formation of or further formation of pathogenic protein aggregates either between, outside or inside neurons, wherein said method comprises contacting said neurons at risk of developing said pathogenic protein aggregates with a protein aggregate inhibiting amount of a 5-trans isomer of GGA, provided that said pathogenic protein aggregates are not related to SBMA.

14. The method of claim 13, wherein said pathogenic protein aggregates from between, outside, and/or inside said neurons.

15. A method for inhibiting the neurotoxicity of β-amyloid peptide by contacting the β-amyloid peptide with an effective amount of 5-trans isomer of GGA.

16. The method of claim 15, wherein the β-amyloid peptide is between or outside of neurons, or is part of the β-amyloid plaque.

17. A method for inhibiting neural death and/or increasing neural activity in a mammal suffering from a neural disease, wherein the etiology of said neural disease comprises formation of protein aggregates which are pathogenic to neurons which method comprises administering to said mammal an amount of a 5-trans isomer of GGA, which will inhibit further pathogenic protein aggregation provided that said pathogenic protein aggregation is not intranuclear.

18. A method for inhibiting neural death and/or increasing neural activity in a mammal suffering from ALS or AD, wherein the etiology of said ALS or AD comprises formation of protein aggregates which are pathogenic to neurons which method comprises administering to said mammal an amount of 5-trans isomer of GGA, which will inhibit further pathogenic protein aggregation provided that said pathogenic protein aggregation is not related to SBMA.

19. The method of claim 19, wherein said amount of GGA alters the pathogenic protein aggregate present into a non-pathogenic form or prevents formation of pathogenic protein aggregates.

20. A method for preventing neural death during seizures in a mammal in need thereof, which method comprises administering a therapeutically effective amount of a 5-trans isomer of GGA.

21. A method comprising one or more of the following steps:
(i) reacting a compound of formula III under halogenation conditions to provide a compound of formula IV; 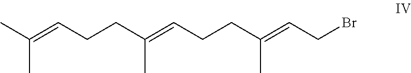
(ii) reacting the compound of formula IV with alkyl acetoacetate under alkylation conditions to provide a compound of formula V: 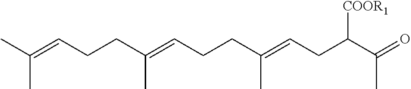
(iii) reacting the compound V under hydrolysis and decarboxylation conditions to provide a compound of formula VI: 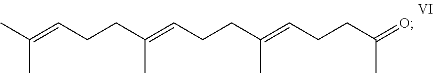
(iv) reacting the compound of formula VI with a compound of formula VII, wherein R₂ and each R₃ independently are alkyl or substituted or unsubstituted aryl, under olefination conditions to selectively provide a compound of formula VIII: 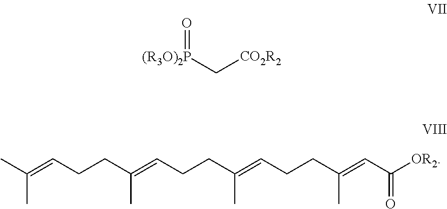
(v) reacting the compound of formula VIII under reduction conditions to provide a compound of formula IX:

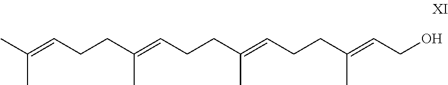

22. The method of claim 21, further comprising repeating steps (i), (ii), and (iii) sequentially using compound of formula IX to provide the compound of formula I, wherein m is 2. 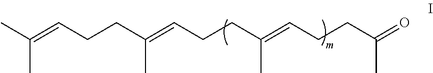
or further comprising repeating steps (i), (ii), (iii), (iv), and (v), sequentially, 1-3 times to produce IB, where m=3.

23. A method comprising reacting a compound of formula VIII: 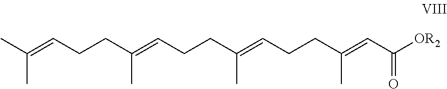
under reduction conditions to provide a compound of formula IX, where n is 2:

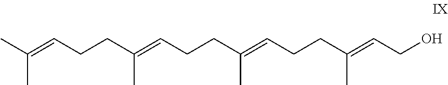

24. A method comprising one or more of the following steps:
(i) reacting under halogenation conditions a compound of formula IIIB: 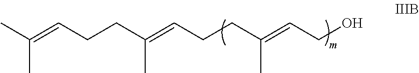
wherein m is 1-3, to provide a compound of formula IVB: 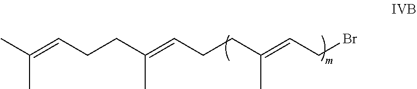
(ii) reacting the compound of formula IVB with alkyl acetoacetates, wherein R₁ is unsubstituted or substituted alkyl under alkylating conditions to provide a compound of formula VB: 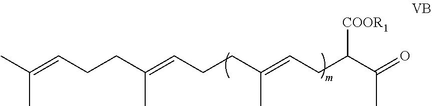
(iii) reacting compound VB under hydrolysis and decarboxylation conditions to provide a compound of formula VIB:

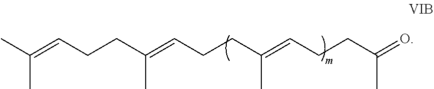

25. A method of reacting a compound of formula VB: 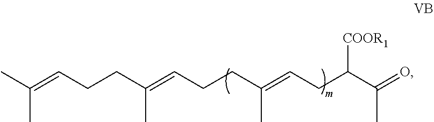
wherein R₁ is alkyl and m is 0-3 under hydrolysis and decarboxylation conditions to provide a compound of formula VB:

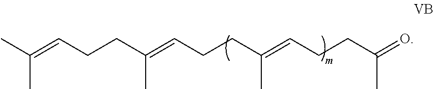

26. A method comprising reacting a ketal compound of formula XII: 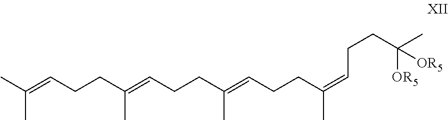
wherein each R₅ independently is C₁-C₆ alkyl, or two R₅ groups together with the oxygen atoms they are attached to form a 5 or 6 membered ring, which ring is optionally substituted with 1-3, preferably 1-2, C₁-C₆ alkyl groups, under hydrolysis conditions to provide a compound of formula II:

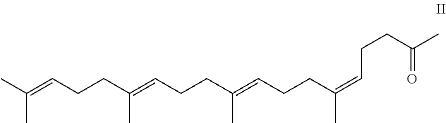

27. A method comprising reacting a compound of formula XI: 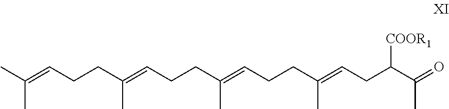
under hydrolysis and subsequently decarboxylation conditions to form a compound of formula I

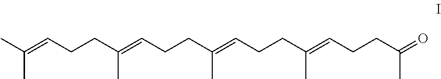

28. A method comprising reacting a compound of formula XIC: 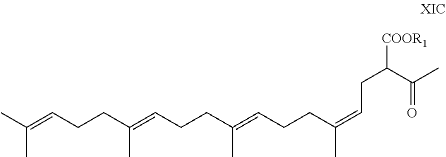
under hydrolysis and subsequent decarboxylation conditions to form the compound of formula II [chemical structure]
